(19) United States
(12) Patent Application Publication
(10) Pub. No.: US 2023/0201384 A1
(43) Pub. Date: Jun. 29, 2023

(54) FGFR3-TARGETED RADIOIMMUNOCONJUGATES AND USES THEREOF

(71) Applicant: Fusion Pharmaceuticals Inc., Hamilton (CA)

(72) Inventors: Eric Steven Burak, Cambridge (CA); Marc Schwabish, Wellesley, MA (US); Natalie Grinshtein, Bothell, WA (US)

(21) Appl. No.: 17/906,857

(22) PCT Filed: Mar. 23, 2021
(86) PCT No.: PCT/US2021/023755

§ 371 Date: Sep. 20, 2022

Related U.S. Application Data
(60) Provisional application No. 62/993,622, filed on Mar. 23, 2020.

(51) Int. Cl. A61K 51/10; A61P 13/10; A61P 35/00

(52) U.S. Cl. CPC A61K 51/1096; A61P 13/10; A61P 35/00

(57) ABSTRACT

Radioimmunoconjugates including a chelating moiety or a metal complex thereof, a linker, and an FGFR3 targeting moiety. Pharmaceutical compositions of such radioimmunoconjugates and methods of treatment for conditions, e.g., cancer, using such pharmaceutical compositions.

### RELATED APPLICATIONS

The present application claims priority to U.S. Provisional Patent Application No. 62/993,622, filed Mar. 23, 2020, the entire contents of which are hereby incorporated by reference for all purposes.

### SEQUENCE LISTING

The present specification makes reference to a Sequence Listing (submitted electronically as a .txt file named “FPI_012_Sequence_Listing.txt” on Mar. 23, 2021). The .txt file was generated on Mar. 22, 2021 and is 8 kb in size. The entire contents of the Sequence Listing are herein incorporated by reference.

### BACKGROUND

Fibroblast growth factors (FGFs) and their receptors (FGFRs) play critical roles during embryonic development, tissue homeostasis and metabolism. In humans, there are 22 FGFs (FGF1-14, FGF16-23) and four FGF receptors with tyrosine kinase domain (FGFR1-4). FGFRs consist of an extracellular ligand binding region, with two or three immunoglobulin-like domains (IgDI-3), a single-pass transmembrane region, and a cytoplasmic, split tyrosine kinase domain. FGFs and their cognate receptors regulate a broad array of cellular processes, including proliferation, differentiation, migration and survival, in a context-dependent manner. FGFRs are overexpressed in many cancer types, often due to mutations that confer constitutive activation.

Aberrantly activated FGFRs have been implicated in specific human malignancies. For example, the t(4; 14) (pl6.3; q32) chromosomal translocation occurs in about 15-20% of multiple myeloma patients, leading to overexpression of FGFR3 and correlates with shorter overall survival. FGFR3 is implicated in conferring chemoresistance to myeloma cell lines in culture, consistent with the poor clinical response of t(4; 14)+ patients to conventional chemotherapy. Overexpression of mutationally activated FGFR3 is sufficient to induce oncogenic transformation in hematopoietic cells and fibroblasts, transgenic mouse models, and murine bone marrow transplantation models. FGFR3-TACC3 (transforming acidic coiled-coil 3) oncogenic fusions have also been observed in a subset of glioblastomas and other cancers, and early data suggests that such tumors may be sensitive to FGFR inhibition. Additionally, genomic alterations that activate FGFR3 are frequent in bladder cancer, including metastatic bladder urothelial carcinoma.

FGFR3 has thus been proposed as a potential therapeutic target for cancer. Several small-molecule inhibitors targeting FGFRs have demonstrated cytotoxicity against FGFR3-positive myeloma cells in culture and in mouse models. However, these small molecules are not selective for FGFR3 and exhibit inhibitory activity toward certain other kinases.

Thus, there remains a need for improved therapeutics (e.g., cancer therapeutics) that can target FGFR3.

### SUMMARY

The present disclosure relates to radioimmunoconjugates that target FGFR3 (e.g., human FGFR3, including wild type and/or mutant FGFR3), pharmaceutical compositions thereof, and methods of treating cancer using such pharmaceutical compositions. In certain embodiments, provided radioimmunoconjugates exhibit an increased excretion rate (e.g., after being administered to a mammal) compared to some currently known radiotherapeutics, while still maintaining therapeutic efficacy. In some embodiments, a faster excretion may limit off-target toxicities by limiting the amount of time that the radioimmunoconjugate stays in a subject. Thus, in some embodiments, provided immunoconjugates exhibit reduced off-target toxicities.

In certain embodiments, provided are radioimmunoconjugate comprising the following structure:

A-L-B (Formula I-a)

wherein A is a chelating moiety or metal complex thereof, wherein B is an FGFR3 targeting moiety, and wherein L is a linker.

In some embodiments, A is a metal complex of a chelating moiety. In some such embodiments, the metal complex comprises a radionuclide. In some embodiments, the radionuclide is an alpha emitter, e.g., an alpha emitter selected from the group consisting of Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), and Terbium-149 (¹⁴⁹Tb), or a progeny thereof. In some embodiments, the radionuclide is ²²⁵Ac or a progeny thereof.

In some embodiments, L is has the structure -L¹-(L²)ₙ-, as shown within Formula I-b:

A-L¹-(L²)ₙ-B Formula I-b

wherein:

A is a chelating moiety or metal complex thereof;

B is an FGFR3 targeting moiety;

L¹ is optionally substituted C₁-C₆ alkyl, optionally substituted C₁-C₆ heteroalkyl, or optionally substituted aryl or heteroaryl;

n is between 1 and 5 (inclusive); and

each L², independently, has the structure:

(—X¹-L³-Z¹—) Formula III

wherein:

X¹ is C═O(NR¹), C═S(NR¹), OC═O(NR¹), NR¹C═O(O), NR¹C═O(NR¹), —CH₂PhC═O(NR¹), —CH₂Ph(NH)C═S(NR¹), O, or NR¹; and each R¹ independently is H, optionally substituted C₁-C₆ alkyl, optionally substituted C₁-C₆ heteroalkyl, or optionally substituted aryl or heteroaryl, in which C₁-C₆ alkyl can be substituted by oxo (═O), heteroaryl, or a combination thereof;

L³ is optionally substituted C₁-C₅₀ alkyl or optionally substituted C₁-C₅₀ heteroalkyl; and

Z¹ is CH₂, C═O, C═S, OC═O, NR¹C═O, or NR¹, wherein R¹ is a hydrogen or optionally substituted C₁-C₆ alkyl or pyrrolidine-2,5-dione.

In some embodiments, L³ is C₅-C₂₀ polyethylene glycol.

In some embodiments, the radioimmunoconjugate or a pharmaceutically acceptable salt thereof comprises the following structure:

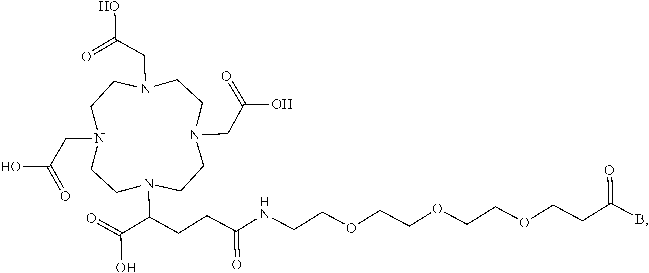

wherein B is an FGFR3 targeting moiety.

In some embodiments, the FGFR3 targeting moiety is at least 100 kDa in size, e.g., at least 150 kDa in size, at least 200 kDa in size, at least 250 kDa in size, or at least 300 kDa in size.

In some embodiments, the FGFR3 targeting moiety is capable of binding to human FGFR3. In some embodiments, the FGFR3 targeting moiety is capable of binding to wild type FGFR3. In some embodiments, the FGFR3 targeting moiety is capable of binding to a mutant FGFR3. In some embodiments, FGFR3 targeting moiety is capable of binding to both wild type and a mutant FGFR3.

In some embodiments, the mutant FGFR3 comprises a point mutation, e.g., a point mutation is associated with cancer. In some embodiments, the point mutant is selected from the group consisting of FGFR3Y375C, FGFR3R248C, FGFR3S249C, FGFR3G372C, FGFR3K652E, FGFR3K652Q, FGFR3K652M, and combinations thereof.

In some embodiments, the mutant FGFR3 comprises an FGFR3 fusion. In some embodiments, the FGFR3 fusion is selected from the group consisting of FGFR3-TACC3, FGFR3-CAMK2A, FGFR3-JAKMOP1, FGFR3-TNIP2, FGFR3-WHSC1, FGFR3-BAIAP2L1, and combinations thereof.

In some embodiments, the FGFR3 targeting moiety comprises an antibody or antigen-binding fragment thereof, e.g., a humanized antibody or antigen-binding fragment thereof.

In some embodiments, the antibody or antigen-binding fragment thereof comprises at least one complementarity determining region (CDR) selected from the group consisting of:

CDR-H1 comprising the amino acid sequence of SEQ ID NO: 1, or an amino acid sequence differing in 1 or 2 amino acids therefrom;

CDR-H2 comprising the amino acid sequence of SEQ ID NO: 2, or an amino acid sequence differing in 1 or 2 amino acids therefrom;

CDR-H3 comprising the amino acid sequence of SEQ ID NO: 3 or 4, or an amino acid sequence differing in 1 or 2 amino acids from SEQ ID NO: 3 or 4;

CDR-L1 comprising the amino acid sequence of SEQ ID NO: 5, or an amino acid sequence differing in 1 or 2 amino acids therefrom;

CDR-L2 comprising the amino acid sequence of SEQ ID NO: 6, or an amino acid sequence differing in 1 or 2 amino acids therefrom; or

CDR-L3 comprising the amino acid sequence of SEQ ID NO: 7, or an amino acid sequence differing in 1 or 2 amino acids therefrom.

In some embodiments, the antibody or antigen-binding fragment thereof comprises at least two CDRs selected from the group consisting of:

CDR-H1 comprising the amino acid sequence of SEQ ID NO: 1, or an amino acid sequence differing in 1 or 2 amino acids therefrom;

CDR-H2 comprising the amino acid sequence of SEQ ID NO: 2, or an amino acid sequence differing in 1 or 2 amino acids therefrom;

CDR-H3 comprising the amino acid sequence of SEQ ID NO: 3 or 4, or an amino acid sequence differing in 1 or 2 amino acids from SEQ ID NO: 3 or 4;

CDR-L1 comprising the amino acid sequence of SEQ ID NO: 5, or an amino acid sequence differing in 1 or 2 amino acids therefrom;

CDR-L2 comprising the amino acid sequence of SEQ ID NO: 6, or an amino acid sequence differing in 1 or 2 amino acids therefrom; or

CDR-L3 comprising the amino acid sequence of SEQ ID NO: 7, or an amino acid sequence differing in 1 or 2 amino acids therefrom.

In some embodiments, the antibody or antigen-binding fragment thereof comprises at least three CDRs selected from the group consisting of:

CDR-H1 comprising the amino acid sequence of SEQ ID NO: 1, or an amino acid sequence differing in 1 or 2 amino acids therefrom;

CDR-H2 comprising the amino acid sequence of SEQ ID NO: 2, or an amino acid sequence differing in 1 or 2 amino acids therefrom;

CDR-H3 comprising the amino acid sequence of SEQ ID NO: 3 or 4, or an amino acid sequence differing in 1 or 2 amino acids from SEQ ID NO: 3 or 4;

CDR-L1 comprising the amino acid sequence of SEQ ID NO: 5, or an amino acid sequence differing in 1 or 2 amino acids therefrom;

CDR-L2 comprising the amino acid sequence of SEQ ID NO: 6, or an amino acid sequence differing in 1 or 2 amino acids therefrom; or

CDR-L3 comprising the amino acid sequence of SEQ ID NO: 7, or an amino acid sequence differing in 1 or 2 amino acids therefrom.

In some embodiments, the antibody or antigen-binding fragment thereof comprises at least four CDRs selected from the group consisting of:

CDR-H1 comprising the amino acid sequence of SEQ ID NO: 1, or an amino acid sequence differing in 1 or 2 amino acids therefrom;

CDR-H2 comprising the amino acid sequence of SEQ ID NO: 2, or an amino acid sequence differing in 1 or 2 amino acids therefrom;

CDR-H3 comprising the amino acid sequence of SEQ ID NO: 3 or 4, or an amino acid sequence differing in 1 or 2 amino acids from SEQ ID NO: 3 or 4;

CDR-L1 comprising the amino acid sequence of SEQ ID NO: 5, or an amino acid sequence differing in 1 or 2 amino acids therefrom;

CDR-L2 comprising the amino acid sequence of SEQ ID NO: 6, or an amino acid sequence differing in 1 or 2 amino acids therefrom; or

CDR-L3 comprising the amino acid sequence of SEQ ID NO: 7, or an amino acid sequence differing in 1 or 2 amino acids therefrom.

In some embodiments, the antibody or antigen-binding fragment thereof comprises at least five CDRs selected from the group consisting of:

CDR-H1 comprising the amino acid sequence of SEQ ID NO: 1, or an amino acid sequence differing in 1 or 2 amino acids therefrom;

CDR-H2 comprising the amino acid sequence of SEQ ID NO: 2, or an amino acid sequence differing in 1 or 2 amino acids therefrom;

CDR-H3 comprising the amino acid sequence of SEQ ID NO: 3 or 4, or an amino acid sequence differing in 1 or 2 amino acids from SEQ ID NO: 3 or 4;

CDR-L1 comprising the amino acid sequence of SEQ ID NO: 5, or an amino acid sequence differing in 1 or 2 amino acids therefrom;

CDR-L2 comprising the amino acid sequence of SEQ ID NO: 6, or an amino acid sequence differing in 1 or 2 amino acids therefrom; or

CDR-L3 comprising the amino acid sequence of SEQ ID NO: 7, or an amino acid sequence differing in 1 or 2 amino acids therefrom.

In some embodiments, the antibody or antigen-binding fragment thereof comprises:

CDR-H1 comprising the amino acid sequence of SEQ ID NO: 1, or an amino acid sequence differing in 1 or 2 amino acids therefrom;

CDR-H2 comprising the amino acid sequence of SEQ ID NO: 2, or an amino acid sequence differing in 1 or 2 amino acids therefrom;

CDR-H3 comprising the amino acid sequence of SEQ ID NO: 3 or 4, or an amino acid sequence differing in 1 or 2 amino acids from SEQ ID NO: 3 or 4;

CDR-L1 comprising the amino acid sequence of SEQ ID NO: 5, or an amino acid sequence differing in 1 or 2 amino acids therefrom;

CDR-L2 comprising the amino acid sequence of SEQ ID NO: 6, or an amino acid sequence differing in 1 or 2 amino acids therefrom; and

CDR-L3 comprising the amino acid sequence of SEQ ID NO: 7, or an amino acid sequence differing in 1 or 2 amino acids therefrom.

In some embodiments, the antibody or antigen-binding fragment thereof comprises:

(i) a heavy chain variable domain comprising at least one CDR selected from the group consisting of:

CDR-H1 comprising the amino acid sequence of SEQ ID NO: 1, or an amino acid sequence differing in 1 or 2 amino acids therefrom;

CDR-H2 comprising the amino acid sequence of SEQ ID NO: 2, or an amino acid sequence differing in 1 or 2 amino acids therefrom; and

CDR-H3 comprising the amino acid sequence of SEQ ID NO: 3 or 4, or an amino acid sequence differing in 1 or 2 amino acids from SEQ ID NO: 3 or 4; and

(ii) a light chain variable domain comprising at least one CDR selected from the group consisting of:

CDR-L1 comprising the amino acid sequence of SEQ ID NO: 5, or an amino acid sequence differing in 1 or 2 amino acids therefrom;

CDR-L2 comprising the amino acid sequence of SEQ ID NO: 6, or an amino acid sequence differing in 1 or 2 amino acids therefrom; and

CDR-L3 comprising the amino acid sequence of SEQ ID NO: 7, or an amino acid sequence differing in 1 or 2 amino acids therefrom.

In some embodiments, the antibody or antigen-binding fragment thereof comprises:

(i) a heavy chain variable domain comprising at least one CDR selected from the group consisting of:

CDR-H1 comprising the amino acid sequence of SEQ ID NO: 1;

CDR-H2 comprising the amino acid sequence of SEQ ID NO: 2; and

CDR-H3 comprising the amino acid sequence of SEQ ID NO: 3 or 4; and

(ii) a light chain variable domain comprising at least one CDR selected from the group consisting of:

CDR-L1 comprising the amino acid sequence of SEQ ID NO: 5;

CDR-L2 comprising the amino acid sequence of SEQ ID NO: 6; and

CDR-L3 comprising the amino acid sequence of SEQ ID NO: 7.

In some embodiments, the antibody or antigen-binding fragment thereof comprises:

(i) a heavy chain variable domain comprising at least two CDRs selected from the group consisting of:

CDR-H1 comprising the amino acid sequence of SEQ ID NO: 1, or an amino acid sequence differing in 1 or 2 amino acids therefrom;

CDR-H2 comprising the amino acid sequence of SEQ ID NO: 2, or an amino acid sequence differing in 1 or 2 amino acids therefrom; and

CDR-H3 comprising the amino acid sequence of SEQ ID NO: 3 or 4, or an amino acid sequence differing in 1 or 2 amino acids from SEQ ID NO: 3 or 4; and

(ii) a light chain variable domain comprising at least two CDRs selected from the group consisting of:

CDR-L1 comprising the amino acid sequence of SEQ ID NO: 5, or an amino acid sequence differing in 1 or 2 amino acids therefrom;

CDR-L2 comprising the amino acid sequence of SEQ ID NO: 6, or an amino acid sequence differing in 1 or 2 amino acids therefrom; and

CDR-L3 comprising the amino acid sequence of SEQ ID NO: 7, or an amino acid sequence differing in 1 or 2 amino acids therefrom.

In some embodiments, the antibody or antigen-binding fragment thereof comprises:

(i) a heavy chain variable domain comprising at least two CDRs selected from the group consisting of:

CDR-H1 comprising the amino acid sequence of SEQ ID NO: 1;

CDR-H2 comprising the amino acid sequence of SEQ ID NO: 2; and

CDR-H3 comprising the amino acid sequence of SEQ ID NO: 3 or 4; and

(ii) a light chain variable domain comprising at least two CDRs selected from the group consisting of:

CDR-L1 comprising the amino acid sequence of SEQ ID NO: 5;

CDR-L2 comprising the amino acid sequence of SEQ ID NO: 6; and

CDR-L3 comprising the amino acid sequence of SEQ ID NO: 7.

In some embodiments, the antibody or antigen-binding fragment thereof comprises:

(i) a heavy chain variable domain comprising:

CDR-H1 comprising the amino acid sequence of SEQ ID NO: 1, or an amino acid sequence differing in 1 or 2 amino acids therefrom;

CDR-H2 comprising the amino acid sequence of SEQ ID NO: 2, or an amino acid sequence differing in 1 or 2 amino acids therefrom; and

CDR-H3 comprising the amino acid sequence of SEQ ID NO: 3 or 4, or an amino acid sequence differing in 1 or 2 amino acids from SEQ ID NO: 3 or 4; and

(ii) a light chain variable domain comprising:

CDR-L1 comprising the amino acid sequence of SEQ ID NO: 5, or an amino acid sequence differing in 1 or 2 amino acids therefrom;

CDR-L2 comprising the amino acid sequence of SEQ ID NO: 6, or an amino acid sequence differing in 1 or 2 amino acids therefrom; and

CDR-L3 comprising the amino acid sequence of SEQ ID NO: 7, or an amino acid sequence differing in 1 or 2 amino acids therefrom.

In some embodiments, the antibody or antigen-binding fragment thereof comprises:

(i) a heavy chain variable domain comprising:

CDR-H1 comprising the amino acid sequence of SEQ ID NO: 1;

CDR-H2 comprising the amino acid sequence of SEQ ID NO: 2; and

CDR-H3 comprising the amino acid sequence of SEQ ID NO: 3 or 4; and

(ii) a light chain variable domain comprising:

CDR-L1 comprising the amino acid sequence of SEQ ID NO: 5;

CDR-L2 comprising the amino acid sequence of SEQ ID NO: 6; and

CDR-L3 comprising the amino acid sequence of SEQ ID NO: 7.

In some embodiments, the antibody or antigen-binding fragment thereof comprises: (i) a heavy chain variable domain having an amino acid sequence with at least 85% identity with the amino acid sequence of SEQ ID NO: 8; and (ii) a light chain variable domain having an amino acid sequence with at least 85% identity with the amino acid sequence of SEQ ID NO: 9.

In some embodiments, the antibody or antigen-binding fragment thereof comprises: (i) a heavy chain variable domain having an amino acid sequence with at least 90% identity with the amino acid sequence of SEQ ID NO: 8; and (ii) a light chain variable domain having an amino acid sequence with at least 90% identity with the amino acid sequence of SEQ ID NO: 9.

In some embodiments, the antibody or antigen-binding fragment thereof comprises: (i) a heavy chain variable domain having an amino acid sequence with at least 95% identity with the amino acid sequence of SEQ ID NO: 8; and (ii) a light chain variable domain having an amino acid sequence with at least 95% identity with the amino acid sequence of SEQ ID NO: 9.

In some embodiments, the antibody or antigen-binding fragment thereof comprises: (i) a heavy chain variable domain comprising the amino acid sequence of SEQ ID NO: 8; and (ii) a light chain variable domain comprising the amino acid sequence of SEQ ID NO: 9.

In some embodiments, the antibody is MFGR1877S (vofatamab).

In some embodiments, after administration of the radioimmunoconjugate or a composition thereof to a mammal, the proportion of radiation excreted by the intestinal routes, renal route, or both routes is at least 2-fold greater than the proportion of radiation excreted by the same route(s) by a comparable mammal that has been administered a reference radioimmunoconjugate.

In some embodiments, after administration of the radioimmunoconjugate or a composition thereof to a mammal, the proportion of radiation excreted by the intestinal routes, renal route, or both routes is at least 3-fold greater than the proportion of radiation excreted by the same route(s) by a comparable mammal that has been administered a reference radioimmunoconjugate.

In some embodiments, A-L- is a metal complex of a compound selected from the group consisting of:

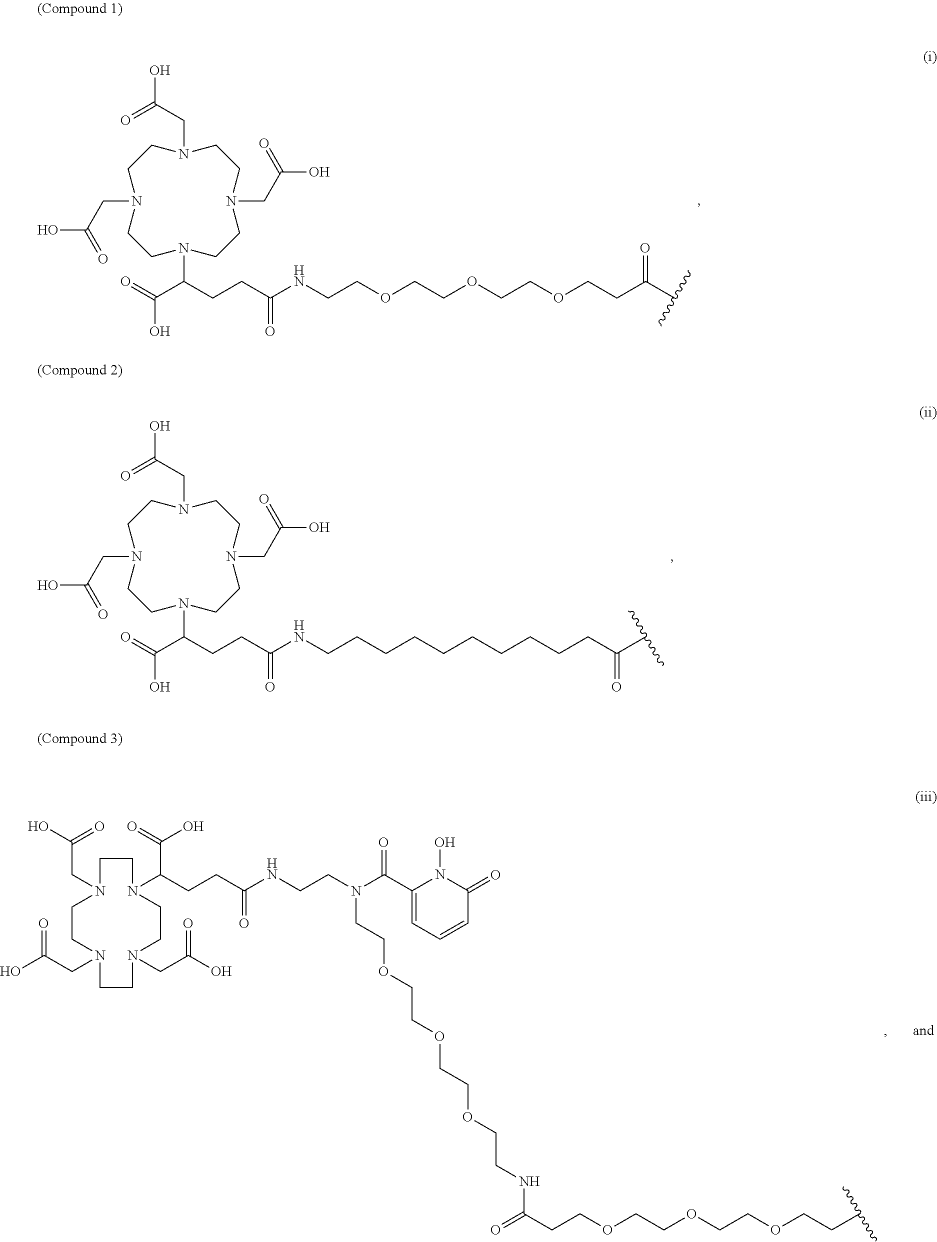 [SMILES: *C(=O)CCOCCOCCOCCNC(=O)CCC(C(=O)O)N1CCN(CC(=O)O)CCN(CC(=O)O)CCN(CC(=O)O)CC1 ; *C(=O)CCCCCCCCCCNC(=O)CCC(C(=O)O)N1CCN(CC(=O)O)CCN(CC(=O)O)CCN(CC(=O)O)CC1 ; *CCOCCOCCOCCC(=O)NCCOCCOCCOCCN(CCNC(=O)CCC(C(=O)O)N1CCN(CC(=O)O)CCN(CC(=O)O)CCN(CC(=O)O)CC1)C(=O)c1cccc(=O)n1O]

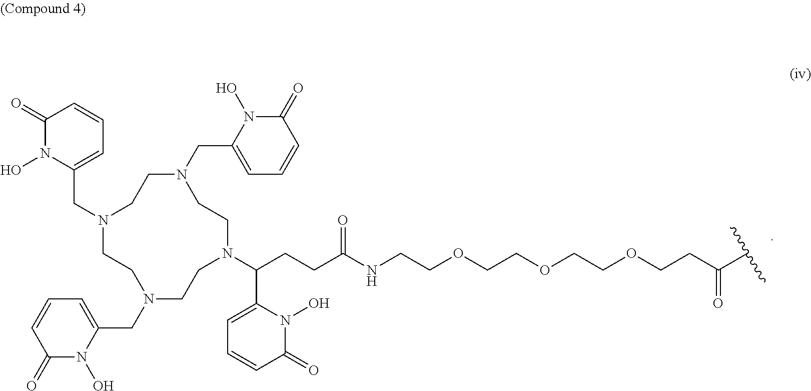 [SMILES: *C(=O)CCOCCOCCOCCNC(=O)CCC(c1cccc(=O)n1O)N1CCN(Cc2cccc(=O)n2O)CCN(Cc2cccc(=O)n2O)CCN(Cc2cccc(=O)n2O)CC1]

In some embodiments, A-L- is a metal complex of:

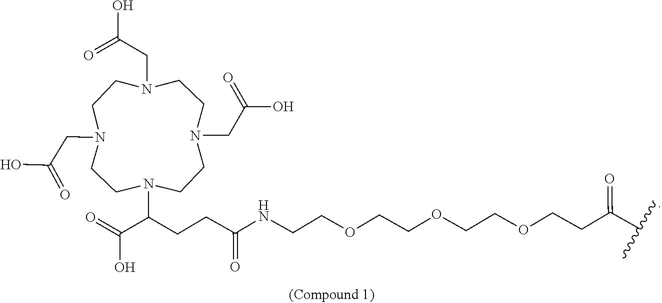 [SMILES: *C(=O)CCOCCOCCOCCNC(=O)CCC(C(=O)O)N1CCN(CC(=O)O)CCN(CC(=O)O)CCN(CC(=O)O)CC1]

In some embodiments, A-L- is a metal complex of

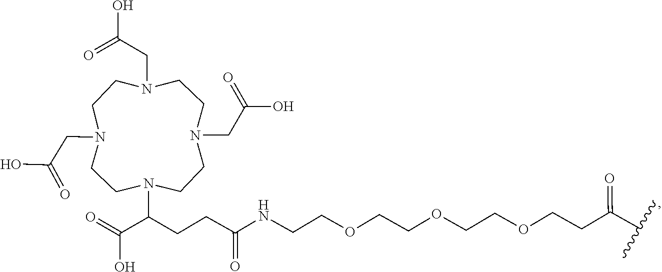 [SMILES: *C(=O)CCOCCOCCOCCNC(=O)CCC(C(=O)O)N1CCN(CC(=O)O)CCN(CC(=O)O)CCN(CC(=O)O)CC1]

and the metal complex comprises a radionuclide, such as an alpha emitter (e.g., Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), and Terbium-149 (¹⁴⁹Tb), or a progeny thereof). In some embodiments, the FGFR3 targeting moiety is an antibody or antigen-binding fragment thereof (e.g., a humanized antibody or antigen-binding fragment thereof).

In some embodiments, A-L- is a metal complex of

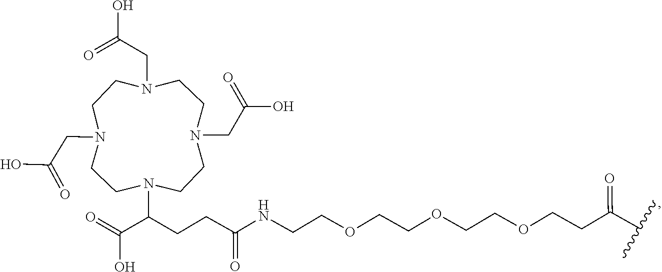 [SMILES: *C(=O)CCOCCOCCOCCNC(=O)CCC(C(=O)O)N1CCN(CC(=O)O)CCN(CC(=O)O)CCN(CC(=O)O)CC1]

the metal complex comprises ²²⁵Ac or a progeny thereof, and the FGFR3 targeting moiety is MFGR1877S (vofatamab) or an antigen-binding fragment thereof. In some embodiments, the FGFR3 targeting moiety is MFGR1877S (vofatamab).

In some embodiments, the radioimmunoconjugate comprising the following structure:

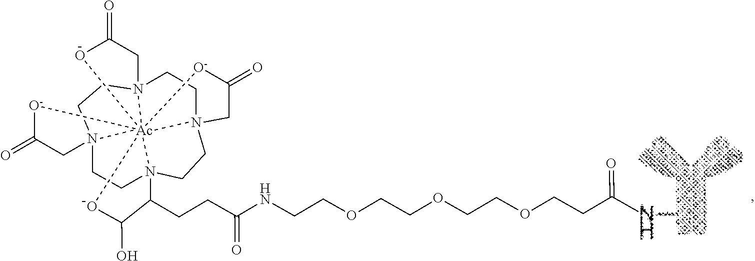

wherein [symbol] is MFGR1877S (vofatamab), wherein the amine group NH-attached to the antibody shown above is from a lysine unit that is part of the antibody.

In certain embodiments, provided are pharmaceutical compositions comprising a radioimmunoconjugate as described herein and a pharmaceutically acceptable carrier.

In certain embodiments, provided are methods of treating cancer, the method comprising administering to a subject in need thereof a pharmaceutical composition comprising an effective amount of a radioimmunoconjugate as described herein.

In some embodiments, the subject is a mammal, e.g., a human.

In some embodiments, the cancer is a solid tumor cancer. In some embodiments, the solid tumor cancer is adrenocortical carcinoma, bladder cancer, breast cancer, cervical cancer, colorectal cancer, endometrial adenocarcinoma, Ewing's sarcoma, gallbladder carcinoma, glioma, head and neck cancer, liver cancer, lung cancer, neuroblastoma, neuroendocrine cancer, pancreatic cancer, prostate cancer, renal cell carcinoma, salivary adenoid cystic cancer, or spermatocytic seminoma. In some embodiments, the solid tumor cancer is bladder cancer. In some embodiments, the solid tumor cancer is glioma. In some embodiments, the solid tumor cancer is neuroblastoma. In some embodiments, the solid tumor cancer is pancreatic cancer. In some embodiments, the solid tumor cancer is breast cancer. In some embodiments, the solid tumor cancer is head and neck cancer. In some embodiments, the solid tumor cancer is live cancer. In some embodiments, the solid tumor cancer is lung cancer.

In some embodiments, the cancer is a non-solid tumor cancer. In some embodiments, the cancer is a liquid cancer or hematologic cancer, e.g., a myeloma (e.g., multiple myeloma), a leukemia, or a lymphoma.

In some embodiments, the pharmaceutical composition is administered systemically. For example, in some embodiments, the pharmaceutical composition is administered parenterally, e.g., intravenously, intraarterially, intraperitoneally, subcutaneously, or intradermally. In some embodiments, the pharmaceutical composition is administered enterically, e.g., trans-gastrointestinally or orally.

In some embodiments, the pharmaceutical composition is administered locally, e.g., by peritumoral injection or by intratumoral injection.

### BRIEF DESCRIPTION OF THE DRAWINGS

FIG. 1A is a schematic depicting the general structure of a conjugate comprising a chelate, a linker, and a targeting moiety.

FIG. 1B is a schematic depicting the structure of [²²⁵Ac]-DOTA-anti-FGFR3, an exemplary radioimmunoconjugate disclosed herein.

FIG. 2 is a schematic depicting the synthesis of the bifunctional chelate, 4-{[11-oxo-11-(2,3,5,6-tetrafluorophenoxy)undecyl]carbamoyl}-2-[4,7,10-tris(carboxymethyl)-1,4,7,10-tetraazacyclododecan-1-yl]butanoic acid (Compound B). Synthesis of Compound B is described in Example 2.

FIG. 3 is a schematic depicting the synthesis of the bifunctional chelate, 4-{[2-(2-{2-[3-oxo-3-(2,3,5,6-tetrafluorophenoxy)propoxy]ethoxy}ethoxy)ethyl]carbamoyl}-2-[4,7,10-tris(carboxymethyl)-1,4,7,10-tetraazacyclododecan-1-yl]butanoic acid (Compound C). Synthesis of Compound C is described in Example 4.

FIGS. 4A-4C are binding curves for unlabeled DOTA-anti-FGFR3 binding to RT4 (FIG. 4A), RT112 (FIG. 4B), and HepG2 (FIG. 4C) FGFR3-positive tumor cells. See Example 16.

FIG. 5 shows a plot representing the results of biodistribution studies in mice bearing RT4 (bladder cancer) xenograft tumors and injected with [¹⁷⁷Lu]-DOTA-anti-FGFR3. Percentage injected dose per gram of tissue (% ID/g) is plotted on the x-axis and is shown for blood, kidney, liver, lung, spleen, skin, tumor, and tail at 4, 24, 48, 96, and 168 hours. See Example 17.

FIG. 6A shows a plot representing the results of biodistribution studies in mice bearing RT112 (bladder cancer) xenograft tumors and injected with [¹⁷⁷Lu]-DOTA-anti-FGFR3. % ID/g is plotted on the x- and is shown for blood, intestine, kidney and adrenal glands, liver and gall bladder, lung, spleen, skin, bladder, urine, and tumor at 4, 24, 48, and 96 hours. See Example 18.

FIG. 6B shows a plot representing the results of biodistribution studies in mice bearing RT112 (bladder cancer) xenograft tumors and injected with [¹⁷⁷Lu]-DOTA-anti-FGFR3 after a pre-dose with cold anti-FGFR3. % ID/g is plotted on the x-axis and is shown for blood, intestine, kidney and adrenal glands, liver and gall bladder, lung, spleen, skin, bladder, urine, and tumor at 4, 24, 48, and 96 hours. Mice received a pre-dose of 100 μg cold (non-radioactive, un-conjugated) anti-FGFR3 antibody 3 hours before receiving [¹⁷⁷Lu]-DOTA-anti-FGFR3. See Example 18.

FIGS. 7A-7C show plots representing the results of biodistribution studies in mice bearing RT112 (bladder cancer) xenograft tumors and co-dosed with cold anti-FGFR3 and [¹⁷⁷Lu]-DOTA-anti-FGFR3. % ID/g is plotted on the x-axis and is shown for blood, intestine, kidney, liver, lung, spleen, skin, bladder, urine, and tumor at 24 and 96 hours. Mice were co-administered 50 μg (FIG. 7A), 100 μg (FIG. 7B), or 200 μg (FIG. 7C) cold anti-FGFR3; cold anti-FGFR3 was administered at the same time as [¹⁷⁷Lu]-DOTA-anti-FGFR3. See Example 19.

FIGS. 8A-8B show plots representing the results of biodistribution studies in mice bearing RT112 (bladder cancer) xenograft tumors and co-dosed with cold anti-FGFR3 and either [¹⁷⁷Lu]-DOTA-anti-FGFR3 (FIG. 8A) or [¹¹¹In]-DOTA-anti-FGFR3 (FIG. 8B). % ID/g is plotted on the x-axis and is shown for blood, intestine, kidney, liver, lung, spleen, skin, bladder, and tumor at 4, 24, 48, 96, and 168 hours. Mice were co-administered 100 μg cold anti-FGFR3 together with [¹⁷⁷Lu]-DOTA-anti-FGFR3. See Example 19.

FIGS. 9A and 9B are plots showing relative tumor volumes (FIG. 9A) and relative body weights (FIG. 9B) in mice who, at the beginning of the experiment, bore RT112 xenograft tumors. Relative tumor volumes (FIG. 9A) and relative body weights (FIG. 9B) are shown at various timepoints after treatment with [²²⁵Ac]-DOTA-anti-FGFR3. Mice were administered a pre-dose of 100 μg cold anti-FGFR3 3 h before dosing with the [²²⁵Ac]-DOTA-anti-FGFR3. See Example 20.

FIGS. 10A and 10B are plots showing relative tumor volumes (FIG. 10A) and relative body weights (FIG. 10B) in mice who, at the beginning of the experiment, bore RT112 xenograft tumors. Relative tumor volumes (FIG. 10A) and relative body weights (FIG. 10B) are shown at various timepoints after treatment with [²²⁵Ac]-DOTA-anti-FGFR3. Mice were co-administered 100 μg cold anti-FGFR3. See Example 21.

### DETAILED DESCRIPTION

Radioimmunoconjugates are designed to target a protein or receptor that is upregulated in a disease state to deliver a radioactive payload to damage and kill cells of interest (radioimmunotherapy). The process of delivering such a payload, via radioactive decay, produces an alpha, beta, or gamma particle or Auger electron that can cause direct effects to DNA (such as single or double stranded DNA breaks) or indirect effects such as by-stander or crossfire effects.

Radioimmunoconjugates typically contain a biological targeting moiety (e.g., an antibody or antigen binding fragment thereof that is capable of specifically binding to human FGFR3), a radioisotope, and a molecule that links the two. Conjugates are formed when a bifunctional chelate is appended to the biological targeting molecule so that structural alterations are minimal while maintaining target affinity. Once radiolabelled, the final radioimmunoconjugate is formed.

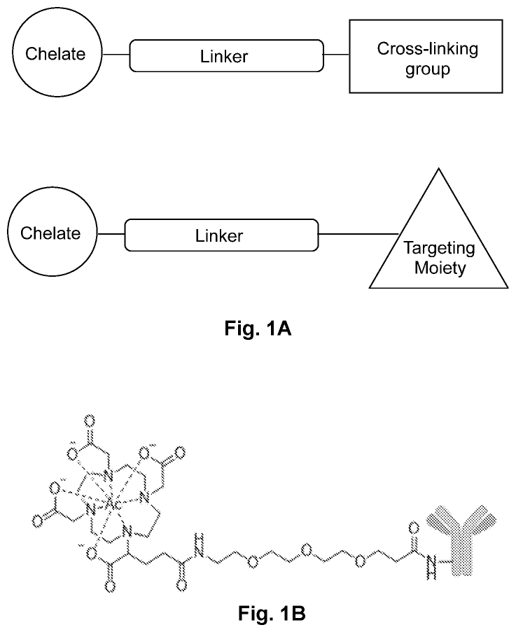

Bifunctional chelates structurally contain a chelate, the linker, and a targeting moiety, e.g., an antibody or antigen-binding fragment thereof (FIG. 1). When developing new bifunctional chelates, most efforts focus around the chelating portion of the molecule. Several examples of bifunctional chelates have been described with various cyclic and acyclic structures conjugated to a targeted moiety. [Bioconjugate Chem. 2000, 11, 510-519; Bioconjugate Chem. 2012, 23, 1029-1039; Mol Imaging Biol. 2011, 13, 215-221, Bioconjugate Chem. 2002, 13, 110-115.]

One of the key factors of developing safe and effective radioimmunoconjugates is maximizing efficacy while minimizing off-target toxicity in normal tissue. While this statement is one of the core tenets of developing new drugs, the application to radioimmunotherapeutics presents new challenges. Radioimmunoconjugates do not need to block a receptor, as needed with a therapeutic antibody, or release the cytotoxic payload intracellularly, as required with an antibody drug conjugate, in order to have therapeutic efficacy. However, the emission of the toxic particle is an event that occurs as a result of first-order (radioactive) decay and can occur at random anywhere inside the body after administration. Once the emission occurs, damage could occur to surrounding cells within the range of the emission leading to the potential of off-target toxicity. Therefore, limiting exposure of these emissions to normal tissue is the key to developing new drugs.

One potential method for reducing off-target exposure is to remove the radioactivity more effectively from the body (e.g., from normal tissue in the body). One mechanism is to increase the rate of clearance of the biological targeting agent. This approach likely requires identifying ways to shorten the half-life of the biological targeting agent, which is not well described for biological targeting agents. Regardless of the mechanism, increasing drug clearance will also negatively impact the pharmacodynamics/efficacy in that the more rapid removal of drug from the body will lower the effective concentration at the site of action, which, in turn, would require a higher total dose and would not achieve the desired results of reducing total radioactive dose to normal tissue.

Other efforts have focused on accelerating the metabolism of the portion of the molecule that contains the radioactive moiety. To this end, some efforts have been made to increase the rate of cleavage of the radioactivity from the biological targeting agents using what have been termed “cleavable linkers”. Cleavable linkers, however, have been taken on different meaning as it relates to radioimmunoconjugates. Cornelissen, et al. has described cleavable linkers as those by which the bifunctional conjugate attaches to the biologic targeting agent through a reduced cysteine, whereas others have described the use of enzyme-cleavable systems that require the co-administration of the radioimmunoconjugate with a cleaving agent/enzyme to release [Mol Cancer Ther. 2013, 12(11), 2472-2482; Methods Mol Biol. 2009, 539, 191-211; Bioconjug Chem. 2003, 14(5), 927-33]. These methods either change the nature of the biological targeting moiety, in the case of the cysteine linkage, or are not practical from a drug development perspective (enzyme cleavable systems) since, in the case of the citations provided, require the administration of two agents.

The present disclosure provides, among other things, radioimmunoconjugates that are more effectively eliminated from the body after catabolism and/or metabolism, while maintain therapeutic efficacy. Disclosed immunoconjugates may, in some embodiments, achieve a reduction of total body radioactivity, for example, by increasing the extent of excretion of the catabolic/metabolic products while maintaining the pharmacokinetics of the intact molecule when compared to known bifunctional chelates. In some embodiments, this reduction in radioactivity results from the clearance of catabolic/metabolic by-products without impacting other in vitro and in vivo properties such as binding specificity (in vitro binding), cellular retention, and tumor uptake in vivo. Thus, in some embodiments, provided radioimmunoconjugates achieve reduced radioactivity in the human body while maintaining on-target activity.

### Definitions

As used herein, “antibody” refers to a polypeptide whose amino acid sequence includes immunoglobulins and fragments thereof which specifically bind to a designated antigen, or fragments thereof. Antibodies in accordance with the present invention may be of any type (e.g., IgA, IgD, IgE, IgG, or IgM) or subtype (e.g., IgA1, IgA2, IgG1, IgG2, IgG3, or IgG4). Those of ordinary skill in the art will appreciate that a characteristic sequence or portion of an antibody may include amino acids found in one or more regions of an antibody (e.g., variable region, hypervariable region, constant region, heavy chain, light chain, and combinations thereof). Moreover, those of ordinary skill in the art will appreciate that a characteristic sequence or portion of an antibody may include one or more polypeptide chains, and may include sequence elements found in the same polypeptide chain or in different polypeptide chains.

As used herein, “antigen-binding fragment” refers to a portion of an antibody that retains the binding characteristics of the parent antibody.

As used herein, the term “bind” or “binding” of a targeting moiety means an at least temporary interaction or association with or to a target molecule, e.g., to human FGFR3 and/or mutant FGFR3, e.g., as described herein.

The terms “bifunctional chelate” or “bifunctional conjugate” as used interchangeably herein, refers to a compound that comprises a chelate or metal complex thereof, a linker, and a targeting moiety e.g., an antibody or antigen-binding fragment thereof. See, e.g., Formula I-a or FIG. 1.

The term “cancer” refers to any cancer caused by the proliferation of malignant neoplastic cells, such as tumors, neoplasms, carcinomas, sarcomas, leukemias, and lymphomas. A “solid tumor cancer” is a cancer comprising an abnormal mass of tissue, e.g., sarcomas, carcinomas, and lymphomas. A “hematological cancer” or “liquid cancer,” as used interchangeably herein, is a cancer present in a body fluid, e.g., lymphomas and leukemias.

The term “chelate” as used herein, refers to an organic compound or portion thereof that can be bonded to a central metal or radiometal atom at two or more points.

The term “conjugate,” as used herein, refers to a molecule that contains a chelating group or metal complex thereof, a linker group, and which optionally contains a targeting moiety, e.g., an antibody or antigen-binding fragment thereof.

As used herein, the term “compound,” is meant to include all stereoisomers, geometric isomers, and tautomers of the structures depicted.

The compounds recited or described herein can be asymmetric (e.g., having one or more stereocenters). All stereoisomers, such as enantiomers and diastereomers, are intended unless otherwise indicated. Compounds discussed in the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis.

As used herein “detection agent” refers to a molecule or atom which is useful in diagnosing a disease by locating the cells containing the antigen. Various methods of labeling polypeptides with detection agents are known in the art. Examples of detection agents include, but are not limited to, radioisotopes and radionuclides, dyes (such as with the biotin-streptavidin complex), contrast agents, luminescent agents (e.g., fluorescein isothiocyanate or FITC, rhodamine, lanthanide phosphors, cyanine, and near IR dyes), and magnetic agents, such as gadolinium chelates.

As used herein, the term “radionuclide,” refers to an atom capable of undergoing radioactive decay (e.g., ³H, ¹⁴C, ¹⁵N, ¹⁸F, ³⁵S, ⁴⁷Sc, ⁵⁵Co, ⁶⁰Cu, ⁶¹Cu, ⁶²Cu, ⁶⁴Cu, ⁶⁷cu ⁷⁵Br, ⁷⁶Br, ⁷⁷Br, ⁸⁹Zr, ⁸⁶Y, ⁸⁷Y, ⁹⁰Y, ⁹⁷Ru, ⁹⁹Tc, ^{99m}Tc, ¹⁰⁵Rh, ¹⁰⁹Pd, ¹¹¹In, ¹²³I, ¹²⁴I, ¹²⁵I, ¹³¹I, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰³Pb, ²¹¹At, ²¹²Pb, ²¹²Bi, ²¹³Bi, ²²³Ra, ²²⁵Ac, ²²⁷Th, ²²⁹Th, ⁶⁶Ga, ⁶⁷Ga, ⁶⁸Ga, ⁸²Rb, ^{117m}Sn, ²⁰¹Tl). The terms radioactive nuclide, radioisotope, or radioactive isotope may also be used to describe a radionuclide. Radionuclides may be used as detection agents, as described herein. In some embodiments, the radionuclide may be an alpha-emitting radionuclide.

The term an “effective amount” of an agent (e.g., any of the foregoing conjugates), as used herein, is that amount sufficient to effect beneficial or desired results, such as clinical results, and, as such, an “effective amount” depends upon the context in which it is being applied. For example, in therapeutic applications, an “effective amount” may be an amount sufficient to cure or at least partially arrest the symptoms of the disorder and its complications, and/or to substantially improve at least one symptom associated with the disease or a medical condition. For example, in the treatment of cancer, an agent or compound that decreases, prevents, delays, suppresses, or arrests any symptom of the disease or condition would be therapeutically effective. A therapeutically effective amount of an agent or compound is not required to cure a disease or condition but may, for example, provide a treatment for a disease or condition such that the onset of the disease or condition is delayed, hindered, or prevented, such that the disease or condition symptoms are ameliorated, or such that the term of the disease or condition is changed. For example, the disease or condition may become less severe and/or recovery is accelerated in an individual. An effective amount may be administered by administering a single dose or multiple (e.g., at least two, at least three, at least four, at least five, or at least six) doses.

The term “immunoconjugate,” as used herein, refers to a conjugate that includes a targeting moiety, such as an antibody (or antigen-binding fragment thereof), nanobody, affibody, or a consensus sequence from Fibronectin type III domain. In some embodiments, the immunoconjugate comprises an average of at least 0.10 conjugates per targeting moiety (e.g., an average of at least 0.2, 0.3, 0.4, 0.5, 0.6, 0.7, 0.8, 0.9, 1, 2, 4, 5, or 8 conjugates per targeting moiety).

The term “radioconjugate,” as used herein, refers to any conjugate that includes a radioisotope or radionuclide, such as any of the radioisotopes or radionuclides described herein.

The term “radioimmunoconjugate,” as used herein, refers to any immunoconjugate that includes a radioisotope or radionuclide, such as any of the radioisotopes or radionuclides described herein.

The term “radioimmunotherapy,” as used herein, refers a method of using a radioimmunoconjugate to produce a therapeutic effect. In some embodiments, radioimmunotherapy may include administration of a radioimmunoconjugate to a subject in need thereof, wherein administration of the radioimmunoconjugate produces a therapeutic effect in the subject. In some embodiments, radioimmunotherapy may include administration of a radioimmunoconjugate to a cell, wherein administration of the radioimmunoconjugate kills the cell. Wherein radioimmunotherapy involves the selective killing of a cell, in some embodiments the cell is a cancer cell in a subject having cancer.

The term “pharmaceutical composition,” as used herein, represents a composition containing a radioimunoconjugate described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, radioprotectants, sorbents, suspending or dispersing agents, sweeteners, or waters of hydration. Exemplary excipients include, but are not limited to: ascorbic acid, histidine, phosphate buffer, butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts of the compounds described here that are, within the scope of sound medical judgment, suitable for use in contact with the tissues of humans and animals without undue toxicity, irritation, or allergic response. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. Salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, among others. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

The term “polypeptide” as used herein refers to a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides can include one or more “non-natural” amino acids or other entities that nonetheless are capable of integrating into a polypeptide chain. In some embodiments, a polypeptide may be glycosylated, e.g., a polypeptide may contain one or more covalently linked sugar moieties. In some embodiments, a single “polypeptide” (e.g., an antibody polypeptide) may comprise two or more individual polypeptide chains, which may in some cases be linked to one another, for example by one or more disulfide bonds or other means.

By “subject” is meant a human or non-human animal (e.g., a mammal).

By “substantial identity” or “substantially identical” is meant a polypeptide sequence that has the same polypeptide sequence, respectively, as a reference sequence, or has a specified percentage of amino acid residues, respectively, that are the same at the corresponding location within a reference sequence when the two sequences are optimally aligned. For example, an amino acid sequence that is “substantially identical” to a reference sequence has at least 50%, 60%, 70%, 75%, 80%, 85%, 90%, 95%, 96%, 97%, 98%, 99%, or 100% identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full-length sequence). Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

As used herein, the term “targeting moiety” refers to any molecule or any part of a molecule that is capable of binding to a given target. The term, “FGFR3 targeting moiety” refers to a targeting moiety that is capable of binding to an FGFR3 molecule, e.g., a human FGFR3, e.g. a wild type or mutant FGFR3.

As used herein, and as well understood in the art, “to treat” a condition or “treatment” of the condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

As used herein, the term “about” or “approximately,” when used in reference to a quantitative value, includes the recited quantitative value itself, unless specifically stated otherwise. As used herein, the term “about” or “approximately” refers to a ±10% variation from the recited quantitative value unless otherwise indicated or inferred from the context.

### Radioimmunoconjugates

In one aspect, the present disclosure provides radioimmunoconjugates having structure of Formula I-a:

A-L-B Formula I-a

wherein A is a chelating moiety or metal complex thereof,

wherein B is a FGFR3 targeting moiety, and

wherein L is a linker.

In some embodiments, the radioimmunoconjugate has or comprises the structure shown in Formula II:

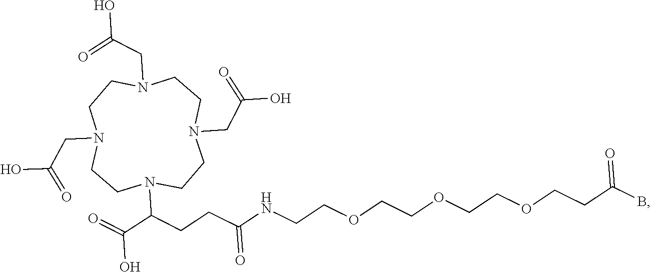

wherein B is the FGFR3 targeting moiety.

In some embodiments, A-L- is a metal complex of a compound selected from the group consisting of

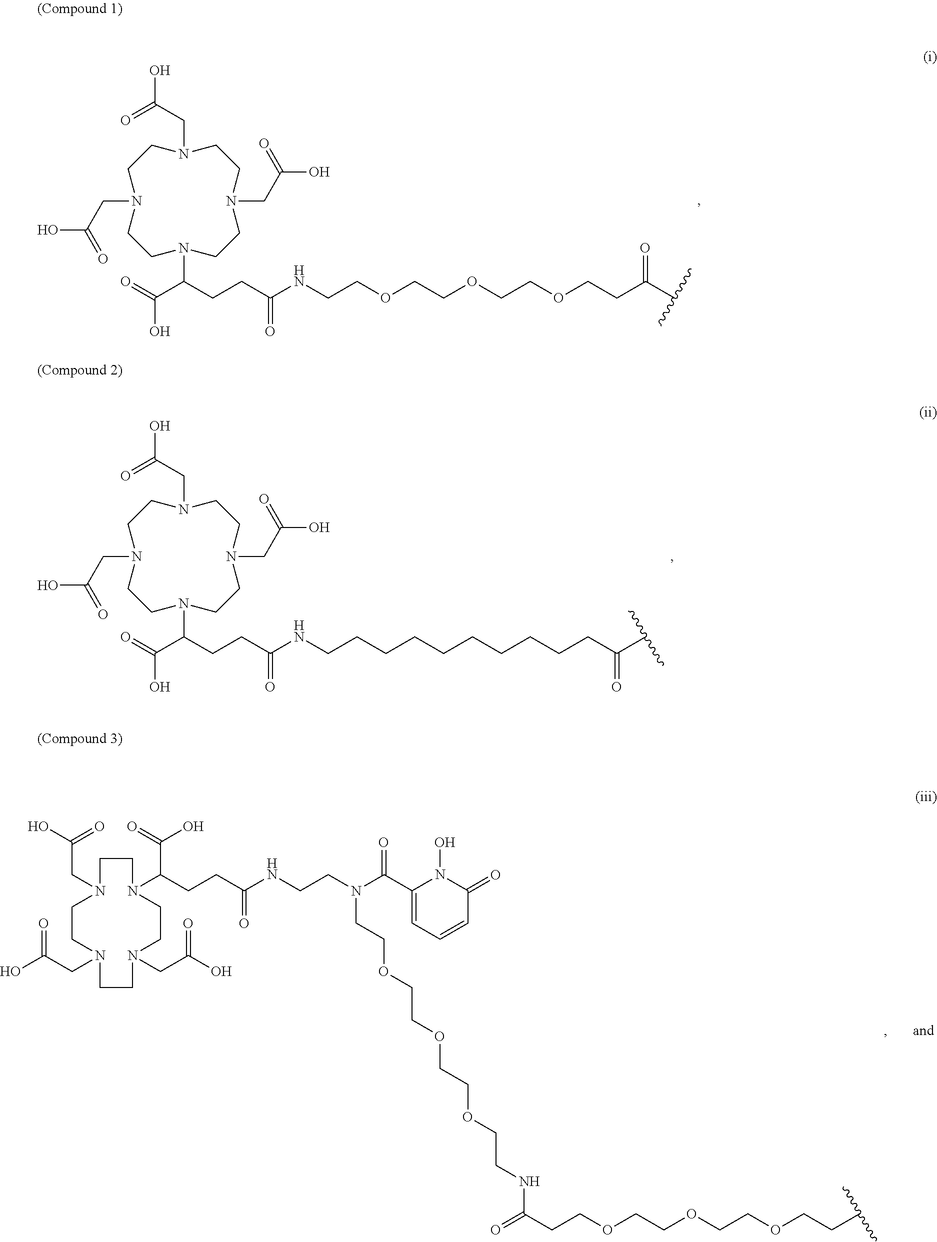 [SMILES: *C(=O)CCOCCOCCOCCNC(=O)CCC(C(=O)O)N1CCN(CC(=O)O)CCN(CC(=O)O)CCN(CC(=O)O)CC1 ; *C(=O)CCCCCCCCCCNC(=O)CCC(C(=O)O)N1CCN(CC(=O)O)CCN(CC(=O)O)CCN(CC(=O)O)CC1 ; *CCOCCOCCOCCC(=O)NCCOCCOCCOCCN(CCNC(=O)CCC(C(=O)O)N1CCN(CC(=O)O)CCN(CC(=O)O)CCN(CC(=O)O)CC1)C(=O)c1cccc(=O)n1O]

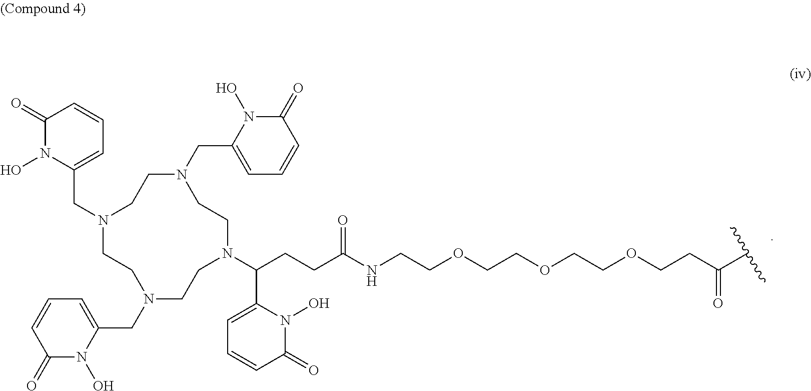 [SMILES: *C(=O)CCOCCOCCOCCNC(=O)CCC(c1cccc(=O)n1O)N1CCN(Cc2cccc(=O)n2O)CCN(Cc2cccc(=O)n2O)CCN(Cc2cccc(=O)n2O)CC1]

In some embodiments, as further described herein, the radioimmunoconjugate comprises a chelating moiety or metal complex thereof, which metal complex may comprise a radionuclide. In some such radiommunoconjugates, the average ratio or median ratio of the chelating moiety to the FGFR3 targeting moiety is eight or less, seven or less, six or less, five or less, four or less, three or less, two or less, or about one. In some radioimmunoconjugates, the average ratio or median ratio of the chelating moiety to the FGFR3 targeting moiety is about one.

In some embodiments, after the radioimmunoconjugate is administered to a mammal, the proportion of radiation (of the total amount of radiation that is administered) that is excreted by the intestinal route, the renal route, or both is greater than the proportion of radiation excreted by a comparable mammal that has been administered a reference radioimmunoconjugate. By “reference immunoconjugate” it is meant a known radioimmunoconjugate that differs from a radioimmunoconjugate described herein at least by (1) having a different linker; (2) having a targeting moiety of a different size and/or (3) lacking a targeting moiety. In some embodiments, the reference radioimmunoconjugate is selected from the group consisting of [⁹⁰Y]-ibritumomab tiuxetan (Zevalin (⁹⁰Y)) and [¹¹¹In]-ibritumomab tiuxetan (Zevalin (¹¹¹In)).

In some embodiments, the proportion of radiation excreted by a given route or set of routes) is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% greater than the proportion of radiation excreted by the same route(s) by a comparable mammal that has been administered a reference radioimmunoconjugate. In some embodiments, the proportion of radiation excreted is at least 1.5-fold, at least 2-fold, at least 2.5-fold, at least 3-fold, at least 3.5 fold, at least 4-fold, at least 4.5 fold, at least 5 fold, at least 6-fold, at least 7-fold, at least 8-fold, at least 9-fold, or at least 10-fold greater than proportion of radiation excreted by a comparable mammal that has been administered a reference radioimmunoconjugate. The extent of excretion can be measured by methods known in the art, e.g., by measuring radioactivity in urine and/or feces and/or by measuring total body radioactivity over a period time. See also, e.g., International Patent Publication WO 2018/024869.

In some embodiments, the extent of excretion is measured at a time period of at least or about 12 hours after administration, at least or about 24 hours after administration, at least or about 2 days after administration, at least or about 3 days after administration, at least or about 4 days after administration, at least or about 5 days after administration, at least or about 6 days after administration, or at least or about 7 days, after administration.

In some embodiments, after a radioimmunoconjugate has been administered to a mammal, the radioimmunoconjugate exhibits decreased off-target binding effects (e.g., toxicities) as compared to a reference conjugate (e.g., a reference immunoconjugate such as a reference radioimmunoconjugate). In some embodiments, this decreased off-target binding effect is a feature of a radioimmunoconjugate that also exhibits a greater excretion rate as described herein.

### Targeting Moieties

Targeting moieties include any molecule or any part of a molecule that is capable of binding to a given target, e.g., FGFR3. In some embodiments, the targeting moiety comprises a protein or polypeptide. In some embodiments, the targeting moiety is selected from the group consisting of antibodies or antigen binding fragments thereof, nanobodies, affibodies, and consensus sequences from Fibronectin type III domains (e.g., Centyrins or Adnectins). In some embodiments, a moiety is both a targeting and a therapeutic moiety, i.e., the moiety is capable of binding to a given target and also confers a therapeutic benefit. In some embodiments, the targeting moiety comprises a small molecule.

In some embodiments, the targeting moiety has a molecular weight of at least 50 kDa, at least 75 kDa, at least 100 kDa, at least 125 kDa, at least 150 kDa, at least 175 kDa, at least 200 kDa, at least 225 kDa, at least 250 kDa, at least 275 kDa, or at least 300 kDa.

Typically, the targeting moiety is capable of binding to FGFR3, e.g., wild type and/or mutant FGFR3. In some embodiments, the targeting moiety is capable of binding to human FGFR3, e.g., wild type and/or mutant human FGFR3.

In some embodiments, the targeting moiety is capable of binding specifically to FGFR3 (e.g., is capable of binding to FGFR3 while exhibiting comparatively little or no binding to other kinases such as other FGFR proteins).

In some embodiments, the targeting moiety is capable of binding to an extracellular region of FGFR3, e.g., the IgD1 region, the IgD2 region, the IgD3 region, the linker region between IgD1 and IgD2, the linker region between IgD2 and IgD3, or the extracellular juxtamembrane domain. In some embodiments, the targeting moiety is capable of binding to the linker region between IgD2 and IgD3. In some embodiments, the targeting moiety is capable of binding to the extracellular juxtamembrane domain.

In some embodiments, the targeting moiety is capable of binding to the IIIb isoform of FGFR3. In some embodiments, the targeting moiety is capable of binding to the IIIc isoform of FGFR3. In some embodiments, the targeting moiety is capable of binding to both the IIIb and IIIc isoforms of FGFR3.

In some embodiments, the targeting moiety is capable of binding to a mutant FGFR3, e.g., a mutant human FGFR3. Some FGFR3 mutations give rise to an unpaired cysteine, which may lead to ligand-independent receptor dimerization and/or constitutive activation. In some embodiments, the mutant FGFR3 is an activated mutant and/or is associated with cancer.

In some embodiments, the targeting moiety is capable of binding to wild type FGFR3 and at least one mutant FGFR3 associated with cancer.

In some embodiments, the mutant FGFR3 comprises a mutation in an extracellular region of FGFR3. For example, in some embodiments, the mutant FGFR3 comprises a mutation in the linker region between IgD2 and IgD3 and/or in the extracellular juxtamembrane region of FGFR3.

In some embodiments, the mutant FGFR3 comprises a mutation in an intracellular region of FGFR3, e.g., a kinase domain, of FGFR3.

In some embodiments, the mutant FGFR3 comprises a point mutation. Non-limiting examples of FGFR3 point mutants associated with cancer include FGFR3^{Y375C}, FGFR3^{R248C}, FGFR3^{S249C}, FGFR3^{G372C}, FGFR3^{K652E}, FGFR3^{K652Q}, FGFR3^{K652M} and combinations thereof.

In some embodiments, the mutant FGFR3 is ligand-dependent (e.g., FGFR3^{G372C} or FGFR3^{Y375C}). In some embodiments, the mutant FGFR3 is constitutively active (e.g., FGFR3^{R248C} or FGFR3^{S249C}). In some embodiments, the mutant FGFR3 is both ligand-dependent and constitutively active (e.g., FGFR3^{K652E}).

In some embodiments, the mutant FGFR3 comprises an FGFR3 fusion, e.g., a constitutively activated and/or oncogenic fusion, such as a fusion that arises from a translocation. For example, FGFR3-TACC3, FGFR3-CAMK2A, FGFR3-JAKMOP1, FGFR3-TNIP2, FGFR3-WHSC1, and FGFR3-BAIAP2L1 (also known as FGFR3-IRTKS) fusions have been associated with cancer.

In some embodiments, the mutant FGFR3 is an amplifying mutation, e.g., comprising increased copy numbers and/or resulting in higher expression relative to a wild type FGFR3.

In some embodiments, the targeting moiety inhibits FGFR3. By “inhibits,” it is meant that the targeting moiety at least partially inhibits one or more functions of FGFR3 (e.g., human FGFR3). In some embodiments, the targeting moiety at least partially inhibits one or more functions of wild type FGFR3, e.g., wild type human FGFR3. In some embodiments, the targeting moiety at least partially inhibits one or more functions of a mutant FGFR3, e.g., mutant human FGFR3.

In some embodiments, targeting moiety blocks ligand binding to FGFR3 and/or receptor dimerization of FGFR3. For example, in some embodiments, a targeting moiety that blocks ligand binding competes with FGF ligands for interaction with the IIIb and/or the IIIc isoforms of FGFR3.

In some embodiments, the targeting moiety impairs signaling downstream of the FGFR3 receptor, e.g., results in decreased phosphorylation and/or protein or transcript levels of one or more downstream mediators of FGFR3 such as FRS2α, AKT, and p44/42 MAPK.

### Antibodies

Antibodies typically comprise two identical light polypeptide chains and two identical heavy polypeptide chains linked together by disulfide bonds. The first domain located at the amino terminus of each chain is variable in amino acid sequence, providing the antibody-binding specificities of each individual antibody. These are known as variable heavy (VH) and variable light (VL) regions. The other domains of each chain are relatively invariant in amino acid sequence and are known as constant heavy (CH) and constant light (CL) regions. Light chains typically comprise one variable region (VL) and one constant region (CL). An IgG heavy chain includes a variable region (VH), a first constant region (CH1), a hinge region, a second constant region (CH₂), and a third constant region (CH₃). In IgE and IgM antibodies, the heavy chain includes an additional constant region (CH₄).

Antibodies suitable for use with the present disclosure can include, for example, monoclonal antibodies, polyclonal antibodies, multispecific antibodies, human antibodies, humanized antibodies, camelid antibodies, chimeric antibodies, single-chain Fvs (scFv), disulfide-linked Fvs (sdFv), and anti-idiotypic (anti-Id) antibodies, and antigen-binding fragments of any of the above. In some embodiments, the antibody or antigen-binding fragment thereof is humanized. In some embodiments, the antibody or antigen-binding fragment thereof is chimeric. Antibodies can be of any type (e.g., IgG, IgE, IgM, IgD, IgA and IgY), class (e.g., IgG1, IgG2, IgG3, IgG4, IgA1 and IgA2) or subclass.

The term “antigen binding fragment” of an antibody, as used herein, refers to one or more fragments of an antibody that retain the ability to specifically bind to an antigen. Examples of binding fragments encompassed within the term “antigen binding fragment” of an antibody include a Fab fragment, a F(ab′)2 fragment, a Fd fragment, a Fv fragment, a scFv fragment, a dAb fragment (Ward et al., (1989) Nature 341:544-546), and an isolated complementarity determining region (CDR). In some embodiments, an “antigen binding fragment” comprises a heavy chain variable region and a light chain variable region. These antibody fragments can be obtained using conventional techniques known to those with skill in the art, and the fragments can be screened for utility in the same manner as are intact antibodies.

Antibodies or antigen-binding fragments described herein can be produced by any method known in the art for the synthesis of antibodies (See, e.g., Harlow et al., Antibodies: A Laboratory Manual, (Cold Spring Harbor Laboratory Press, 2nd ed. 1988); Brinkman et al., 1995, J. Immunol. Methods 182:41-50; WO 92/22324; WO 98/46645). Chimeric antibodies can be produced using the methods described in, e.g., Morrison, 1985, Science 229:1202, and humanized antibodies by methods described in, e.g., U.S. Pat. No. 6,180,370.

Additional antibodies described herein are bispecific antibodies and multivalent antibodies, as described in, e.g., Segal et al., J. Immunol. Methods 248:1-6 (2001); and Tutt et al., J. Immunol. 147: 60 (1991), or any of the molecules described herein.

“Avimer” relates to a multimeric binding protein or peptide engineered using, for example, in vitro exon shuffling and phage display. Multiple binding domains are linked, resulting in greater affinity and specificity compared to single epitope immunoglobin domains.

“Nanobodies” are antibody fragments consisting of a single monomeric variable antibody domain. Nanobodies may also be referred to as single-domain antibodies. Like antibodies, nanobodies are capable of binding selectively to a specific antigen. Nanobodies may be heavy-chain variable domains or light chain domains. Nanobodies may occur naturally or be the product of biological engineering. Nanobodies may be biologically engineered by site-directed mutagenesis or mutagenic screening (e.g., phage display, yeast display, bacterial display, mRNA display, ribosome display). “Affibodies” are polypeptides or proteins engineered to bind to a specific antigen. As such, affibodies may be considered to mimic certain functions of antibodies.

Affibodies may be engineered variants of the B-domain in the immunoglobulin-binding region of staphylococcal protein A. Affibodies may be engineered variants of the Z-domain, a B-domain that has lower affinity for the Fab region. Affibodies may be biologically engineered by site-directed mutagenesis or mutagenic screening (e.g., phage display, yeast display, bacterial display, mRNA display, ribosome display).

Affibody molecules showing specific binding to a variety of different proteins (e.g. insulin, fibrinogen, transferrin, tumor necrosis factor-α, IL-8, gp120, CD28, human serum albumin, IgA, IgE, IgM, HER2 and EGFR) have been generated, demonstrating affinities (Kd) in the M to μM range. “Diabodies” are antibody fragments with two antigen-binding sites that may be bivalent or bispecific. See for example Hudson et al., (2003). Single-chain antibodies are antibody fragments comprising all or a portion of the heavy chain variable domain or all, or a portion of the light chain variable domain of an antibody. Antibody fragments can be made by various techniques including but not limited to proteolytic digestion of an intact antibody as well as production by recombinant hosts (e.g., E. coli or phage) as described herein.

In certain embodiments, the antibody or antigen-binding fragment thereof is a multispecific, e.g. bispecific. Multispecific antibodies (or antigen-binding fragments thereof) include monoclonal antibodies (or antigen-binding fragments thereof) that have binding specificities for at least two different sites.

In certain embodiments, amino acid sequence variants of antibodies or antigen-binding fragments thereof are contemplated; e.g., variants that are capable of binding to human FGFR3 and/or a mutant FGFR3 (such as a mutant FGFR3 associated with cancer). For example, it may be desirable to improve the binding affinity and/or other biological properties of the antibody or antigen-binding fragment thereof. Amino acid sequence variants of an antibody or antigen-binding fragment thereof may be prepared by introducing appropriate modifications into the nucleotide sequence encoding the antibody or antigen-binding fragment thereof, or by peptide synthesis. Such modifications include, for example, deletions from and/or insertions into and/or substitutions of residues within the amino acid sequences of the antibody or antigen-binding fragment thereof. Any combination of deletion, insertion and substitution can be made to arrive at the final construct, provided that the final construct possesses desired characteristics, e.g. antigen binding.

In some embodiments, the antibody or antigen binding fragment thereof is an inhibitory antibody (also called “antagonistic antibody”) or antigen-binding fragment thereof, e.g., the antibody or antigen binding fragment thereof at least partially inhibits one or more functions of the target molecule (e.g., FGFR3) as explained further herein.

Non-limiting examples of inhibitory antibodies include humanized monoclonal antibodies such as MFGR1877S (CAS No. 1312305-12-6; Genentech) (a human monoclonal antibody also known as vofatamab, and whose lyophilized form is also known as B-701 or R3Mab); PRO-001 (Prochon); PRO-007 (Fibron); IMC-D 11 (Imclone); and AV-370 (Aveo Pharmaceuticals). (See, e.g., U.S. Pat. Nos. 8,410,250; 10,208,120; and International Patent Publication Nos. WO2002102972A2, WO2002102973A2, WO2007144893A2, WO2010002862A2, and WO2010048026A2.)

In some embodiments, the antibody or antigen binding fragment thereof is an agonistic antibody (also known as stimulatory antibody).

In some embodiments, the antibody or antigen biding fragment thereof is neither agonistic or antagonistic, or has not been characterized as either agonistic or antagonistic.

Additional known FGFR3 antibodies include, for example, mouse monoclonal antibodies such as, for example, 1G6, 6G1, and 15B2 from Genentech (See, e.g., U.S. Pat. No. 8,410,250), B9 (Sc-13121) (Santa Cruz Biotechnology), MAB766 (clone 136334) (R&D systems), MAB7661 (clone 136318) (R&D systems), and OTI1B10 (OriGene); rabbit polyclonal antibodies such as, for example, ab10651 (Abcam); and rabbit monoclonal antibodies such as C51F2 (catalog number 4574) (Cell Signaling Technology).

In certain embodiments of the present disclosure, the antibody or antigen-binding fragment thereof comprises specific heavy chain complementarity determining regions CDR-H1, CDR-H2 and/or CDR-H3 as described herein. In some embodiments, the complementarity determining regions (CDRs) of the antibody or antigen-binding fragment thereof are flanked by framework regions. A heavy or light chain of an antibody or antigen-binding fragment thereof containing three CDRs typically contains four framework regions.

In some embodiments, the heavy chain variable region of the FGFR3 antibody or antibody-binding fragment thereof comprises one, two, or three complementarity determining regions (CDRs) CDR-H1, CDR-H2, and/or CDR-H3, with amino acid sequences shown below, or CDR region(s) having an amino acid sequence(s) differing in 1 or 2 amino acids therefrom:

| | CDR-H1: |
|---|---|
| (SEQ ID NO: 1) | |
| | GFTFTSTGIS |
| | CDR-H2: |
| (SEQ ID NO: 2) | |
| | GRIYPTSGSTNYADSV |
| | CDR-H3: |
| (SEQ ID NO: 3) | |
| | TYGIYDLYVDYTEYVMDY |
| | or |
| (SEQ ID NO: 4) | |
| | ARTYGIYDLYVDYTEYVMDY |

In some embodiments, the light chain variable region of the FGFR3 antibody or antibody-binding fragment thereof comprises one, two, or three complementarity determining regions (CDRs) CDR-L1, CDR-L2, and/or CDR-L3. with amino acid sequences as shown below, or CDR region(s) having an amino acid sequence(s) differing in 1 or 2 amino acids therefrom:

| | CDR-L1: |
|---|---|
| (SEQ ID NO: 5) | |
| | RASQDVDTSLA |
| | CDR-L2: |
| (SEQ ID NO: 6) | |
| | SASFLYS |
| | CDR-L3: |
| (SEQ ID NO: 7) | |
| | QQSTGHPQT |

In some embodiments, the antibody or antigen-binding fragment thereof comprises:

(i) a heavy chain comprising:

a heavy chain complementarity determining region 1 (CDR-H1) having the amino acid sequence as shown in SEQ ID NO: 1 or an amino acid sequence differing in 1 or 2 amino acids therefrom,

a heavy chain complementarity determining region 2 (CDR-H2) having the amino acid sequence as shown in SEQ ID NO: 2 or an amino acid sequence differing in 1 or 2 amino acids therefrom, and

a heavy chain complementarity determining region 3 (CDR-H3) having the amino acid sequence as shown in SEQ ID NO: 3 or 4 or an amino acid sequence differing in 1 or 2 amino acids therefrom, and

(ii) a light chain comprising:

a light chain complementarity determining region 1 (CDR-L1) having the amino acid sequence as shown in SEQ ID NO: 5 or an amino acid sequence differing in 1 or 2 amino acids therefrom,

a light chain complementarity determining region 2 (CDR-L2) having the amino acid sequence as shown in SEQ ID NO: 6 or an amino acid sequence differing in 1 or 2 amino acids therefrom, and

a light chain complementarity determining region 3 (CDR-L3) having the amino acid sequence as shown in SEQ ID NO: 7 or an amino acid sequence differing in 1 or 2 amino acids therefrom;

or a monoclonal antibody recognizing the same epitope on FGFR3.

In some embodiments, the antibody or antigen-binding fragment thereof has CDR sequences having amino acid sequences of SEQ ID NOs: 1, 2, 3, 5, 6, and 7 without any variation. For example, in some embodiments, the antibody or antigen-binding fragment thereof comprises heavy chain complementary determining regions CDR-H1, CDR-H2, and CDR-H3 having the amino acid sequences of SEQ ID NOs: 1, 2, and 3, and the chain complementarity determining regions CDR-L1, CDR-L2, and CDR-L3 having the amino acid sequences of SEQ ID NOs: 5, 6, and 7.

In some embodiments, the antibody or antigen-binding fragment thereof has CDR sequences having amino acid sequences of SEQ ID NOs: 1, 2, 4, 5, 6, and 7 without any variation. For example, in some embodiments, the antibody or antigen-binding fragment thereof comprises heavy chain complementary determining regions CDR-H1, CDR-H2, and CDR-H3 having the amino acid sequences of SEQ ID NOs: 1, 2, and 4, and the chain complementarity determining regions CDR-L1, CDR-L2, and CDR-L3 having the amino acid sequences of SEQ ID NOs: 5, 6, and 7.

In some embodiments, the heavy chain variable region of the FGFR3 antibody or antigen-binding fragment thereof comprises an amino acid sequence of SEQ ID NO: 9 or an amino acid sequence differing in 1, 2, 3, or 4 amino acids therefrom, or an amino acid sequence having at least 85%, at least 90%, at least 95%, at least 97%, or at least 99% identical to SEQ ID NO: 8:

(SEQ ID NO: 8)

EVQLVESGGG LVQPGGSLRL SCAASGFTFT STGISWVRQ
APGKGLEWVGR IYPTSGSTNY ADSVKGRFTI SADTSKNTAY
LQMNSLRAED TAVYYCARTY GIYDLYVDYT EYVMDYWGQG
TLVTVSSAST KGPSVFPLAP SSKSTSGGTA ALGCLVKDYF
PEPVTVSWNS GALTSGVHTF PAVLQSSGLY SLSSVVTVPS
SSLGTQTYIC NVNHKPSNTK VDKKVEPKSC DKTHTCPPCP
APELLGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLTCLVK GFYPSDIAVE WESNGQPENN
YKTTPPVLDS DGSFFLYSKL TVDKSRWQQG NVFSCSVMHE
ALHNHYTQKS LSLSPGK

In some embodiments, the light chain variable region of the FGFR3 antibody or antigen-binding fragment thereof comprises an amino acid sequence of SEQ ID NO: 9 or an amino acid sequence differing in 1, 2, 3, or 4 amino acids therefrom, or an amino acid sequence having at least 85%, at least 90%, at least 95%, at least 97%, or at least 99% identical to SEQ ID NO: 9:

(SEQ ID NO: 9)

DIQMTQSPSS LSASVGDRVT ITCRASQDVD TSLAWYKQKP
GKAPKLLIYS ASFLYSGVPS RFSGSGSGTD FTLTISSLQP
EDFATYYCQQ STGHPQTFGQ GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

In some embodiments, the FGFR3 targeting moiety is MFGR1877S (vofatamab) or an antigen-binding fragment thereof.

In some embodiments, the FGFR3 antibody or antigen-binding fragment thereof is a humanized antibody or antigen-binding fragment thereof. In certain embodiments, the antibody or antigen-binding fragment thereof has a dissociation constant (Kd) of ≤1 μM, ≤100 nM, ≤10 nM, ≤1 nM, ≤0.1 nM, ≤0.01 nM, or ≤0.001 nM. In some embodiments, the antibody or antigen-binding fragment thereof has a dissociation constant (Kd) of between 1 nM and 10 nM (inclusive of endpoints) or between 0.1 nM and 1 nM (inclusive of endpoints).

In one embodiment, Kd is measured by a radio-labeled antigen binding assay (Radioimmunoassay, RIA) performed with the Fab version of an antibody or antigen-binding fragment thereof of interest and its antigen.

According to another embodiment, Kd is measured using surface plasmon resonance assays with immobilized antigen. In some embodiments, the antibodies or antigen-binding fragments thereof are human monoclonal antibodies directed against an epitope of human FGFR3 as described herein.

The antibody or antigen-binding fragment thereof may be any antibody or antigen-binding fragment thereof of natural and/or synthetic origin, e.g. an antibody of mammalian origin. In some embodiments, the constant domain, if present, is a human constant domain. In some embodiments, the variable domain is a mammalian variable domain, e.g., a humanized or a human variable domain.

In some embodiments, antibodies used in accordance with this disclosure are monoclonal antibodies. In some embodiments, antibodies are recombinant murine antibodies, chimeric, humanized or fully human antibodies, multispecific antibodies(e.g., bispecific antibodies), or antigen-binding fragments thereof.

In some embodiments, are further coupled to other moieties for, e.g., drug targeting and imaging applications.

In some embodiments, e.g., for diagnostic purposes, the antibody or antigen-binding fragment thereof is labelled, i.e. coupled to a labelling group. Non-limiting examples of suitable labels include radioactive labels, fluorescent labels, suitable dye groups, enzyme labels, chromogenes, chemiluminescent groups, biotinyl groups, predetermined polypeptide epitopes recognized by a secondary reporter etc. In some embodiments, one or more labels are covalently bound to the antibody or antigen-binding fragment thereof.

Those labelled antibodies or antigen-binding fragments thereof (also referred to as “antibody conjugates”) may in particular be used in immunohistochemistry assays or for molecular imaging in vivo.

In some embodiments, e.g., for therapeutic purposes, the antibody or antigen-binding fragment thereof is further conjugated with an effector group, in particular, a therapeutic effector group such as a cytotoxic agent or a radioactive group agent.

### Polypeptides

Polypeptides include, for example, any of a variety of hematologic agents (including, for instance, erythropoietin, blood-clotting factors, etc.), interferons, colony stimulating factors, antibodies, enzymes, and hormones. The identity of a particular polypeptide is not intended to limit the present disclosure, and any polypeptide of interest can be a polypeptide in the present methods.

A reference polypeptide described herein can include a target-binding domain that is capable of binding to a target of interest (e.g., is capable of binding to an antigen, e.g., FGFR3). For example, a polypeptide, such as an antibody, can bind to a transmembrane polypeptide (e.g., receptor) or ligand (e.g., a growth factor).

### Modified Polypeptides

Polypeptides suitable for use with compositions and methods of the present disclosure may have a modified amino acid sequence. Modified polypeptides may be substantially identical to the corresponding reference polypeptide (e.g., the amino acid sequence of the modified polypeptide may have at least 50%, 60%, 70%, 75%, 80%, 85%, 90%, 95%, 96%, 97%, 98%, 99%, or 100% identity to the amino acid sequence of the reference polypeptide). In certain embodiments, the modification does not destroy significantly a desired biological activity (e.g., binding to FGFR3). The modification may reduce (e.g., by at least 5%, 10%, 20%, 25%, 35%, 50%, 60%, 70%, 75%, 80%, 90%, or 95%), may have no effect, or may increase (e.g., by at least 5%, 10%, 25%, 50%, 100%, 200%, 500%, or 1000%) the biological activity of the original polypeptide. The modified polypeptide may have or may optimize a characteristic of a polypeptide, such as in vivo stability, bioavailability, toxicity, immunological activity, immunological identity, and conjugation properties.

Modifications include those by natural processes, such as post-translational processing, or by chemical modification techniques known in the art. Modifications may occur anywhere in a polypeptide including the polypeptide backbone, the amino acid side chains and the amino- or carboxy-terminus. The same type of modification may be present in the same or varying degrees at several sites in a given polypeptide, and a polypeptide may contain more than one type of modification. Polypeptides may be branched as a result of ubiquitination, and they may be cyclic, with or without branching. Cyclic, branched, and branched cyclic polypeptides may result from post-translational natural processes or may be made synthetically. Other modifications include pegylation, acetylation, acylation, addition of acetomidomethyl (Acm) group, ADP-ribosylation, alkylation, amidation, biotinylation, carbamoylation, carboxyethylation, esterification, covalent attachment to flavin, covalent attachment to a heme moiety, covalent attachment of a nucleotide or nucleotide derivative, covalent attachment of drug, covalent attachment of a marker (e.g., fluorescent or radioactive), covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination.

A modified polypeptide can also include an amino acid insertion, deletion, or substitution, either conservative or non-conservative (e.g., D-amino acids, desamino acids) in the polypeptide sequence (e.g., where such changes do not substantially alter the biological activity of the polypeptide). In particular, the addition of one or more cysteine residues to the amino or carboxy-terminus of a polypeptide herein can facilitate conjugation of these polypeptides by, e.g., disulfide bonding. For example, a polypeptide can be modified to include a single cysteine residue at the amino-terminus or a single cysteine residue at the carboxy-terminus. Amino acid substitutions can be conservative (i.e., wherein a residue is replaced by another of the same general type or group) or non-conservative (i.e., wherein a residue is replaced by an amino acid of another type). In addition, a naturally occurring amino acid can be substituted for a non-naturally occurring amino acid (i.e., non-naturally occurring conservative amino acid substitution or a non-naturally occurring non-conservative amino acid substitution).

Polypeptides made synthetically can include substitutions of amino acids not naturally encoded by DNA (e.g., non-naturally occurring or unnatural amino acid). Examples of non-naturally occurring amino acids include D-amino acids, N-protected amino acids, an amino acid having an acetylaminomethyl group attached to a sulfur atom of a cysteine, a pegylated amino acid, the omega amino acids of the formula NH₂(CH₂)ₙCOOH wherein n is 2-6, neutral nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties.

Analogs may be generated by substitutional mutagenesis and retain the biological activity of the original polypeptide. Examples of substitutions identified as “conservative substitutions” are shown in Table 1. If such substitutions result in a change not desired, then other type of substitutions, denominated “exemplary substitutions” in Table 1, or as further described herein in reference to amino acid classes, are introduced and the products screened.

TABLE 1
Amino acid substitutions

| Original | Exemplary | Conservative |
|---|---|---|
| residue | substitution | substitution |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, norleucine | Leu |

Substantial modifications in function or immunological identity are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, and/or (c) the bulk of the side chain.

### Chelating Moiety or Metal Complex Thereof

### Chelating Moieties

Examples of suitable chelating moieties include, but are not limited to, DOTA (1,4,7,10-tetraazacyclododecane-1,4,7,10-tetraacetic acid), DOTMA (1R,4R,7R,10R)-α, α′, α″, α′″-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DOTPA (1,4,7,10-tetraazacyclododecane-1,4,7,10-tetra propionic acid), DO3AM-acetic acid (2-(4,7,10-tris(2-amino-2-oxoethyl)-1,4,7,10-tetraazacyclododecan-1-yl)acetic acid), DOTA-GA anhydride (2,2′,2″-(10-(2,6-dioxotetrahydro-2H-pyran-3-yl)-1,4,7,10-tetraazacyclododecane-1,4,7-triyl)triacetic acid, DOTP (1,4,7,10-tetraazacyclododecane-1,4,7,10-tetra(methylene phosphonic acid)), DOTMP (1,4,6,10-tetraazacyclodecane-1,4,7,10-tetramethylene phosphonic acid, DOTA-4AMP (1,4,7,10-tetraazacyclododecane-1,4,7,10-tetrakis(acetamido-methylenephosphonic acid), CB-TE2A (1,4,8,11-tetraazabicyclo[6.6.2]hexadecane-4,11-diacetic acid), NOTA (1,4,7-triazacyclononane-1,4,7-triacetic acid), NOTP (1,4,7-triazacyclononane-1,4,7-tri(methylene phosphonic acid), TETPA (1,4,8,11-tetraazacyclotetradecane-1,4,8,11-tetrapropionic acid), TETA (1,4,8,11-tetraazacyclotetradecane-1,4,8,11-tetra acetic acid), HEHA (1,4,7,10,13,16-hexaazacyclohexadecane-1,4,7,10,13,16-hexaacetic acid), PEPA (1,4,7,10,13-pentaazacyclopentadecane-N,N′,N″,N′″, N″″-pentaacetic acid), H₄octapa (N,N′-bis(6-carboxy-2-pyridylmethyl)-ethylenediamine-N,N′-diacetic acid), H₂dedpa (1,2-[[6-(carboxy)-pyridin-2-yl]-methylamino]ethane), H₆phospa (N,N′-(methylenephosphonate)-N,N′-[6-(methoxycarbonyl)pyridin-2-yl]-methyl-1,2-diaminoethane), TTHA (triethylenetetramine-N,N,N′,N″,N′″, N′″-hexaacetic acid), DO2P (tetraazacyclododecane dimethanephosphonic acid), HP-DO3A (hydroxypropyltetraazacyclododecanetriacetic acid), EDTA (ethylenediaminetetraacetic acid), Deferoxamine, DTPA (diethylenetriaminepentaacetic acid), DTPA-BMA (diethylenetriaminepentaacetic acid-bismethylamide), octadentate-HOPO (octadentate hydroxypyridinones), or porphyrins.

In some embodiments, radioimmunoconjugates comprise a metal complex of a chelating moiety. For example, chelating groups may be used in metal chelate combinations with metals, such as manganese, iron, and gadolinium and isotopes (e.g., isotopes in the general energy range of 60 to 10,000 keV), such as any of the radioisotopes and radionuclides discussed herein.

In some embodiments, chelating moieties are useful as detection agents, and radioimmunoconjugates comprising such detectable chelating moieties can therefore be used as diagnostic or theranostic agents.

### Radioisotopes and Radionuclides

In some embodiments, the metal complex comprises a radionuclide. Examples of suitable radioisotopes and radionuclides include, but are not limited to, ³H, ¹⁴C, ¹⁵N, ¹⁸F, ³⁵S, ⁴⁷Sc, ⁵⁵Co, ⁶⁰Cu, ⁶¹Cu, ⁶²Cu, ⁶⁴Cu, ⁶⁶Ga, ⁶⁷Ga, ⁶⁷Cu, ⁶⁸Ga, ⁷⁵Br, ⁷⁶Br, ⁷⁷Br, ⁸²Rb, ⁸⁹Zr, ⁸⁶Y, ⁸⁷Y, ⁹⁰Y, ⁹⁷Ru, ⁹⁹Tc, ^{99m}Tc, ¹⁰⁵Rh, ¹⁰⁹Pd, ¹¹¹In, ¹²³I, ¹²⁴I, ¹²⁵I, ¹³¹I, ¹⁴⁹Pm, ¹⁴⁹Tb, ¹⁵³Sm, ¹⁶⁶Ho, ¹⁷⁷Lu, ^{117m}Sn, ¹⁸⁶Re, ¹⁸⁸Re, ¹⁹⁸Au, ¹⁹⁹Au, ²⁰¹Tl, ²⁰³Pb, ²¹¹At, ²¹²Pb, ²¹²Bi, ²¹³Bi, ²²³Ra, ²²⁵Ac, ²²⁷Th, and ²²⁹Th.

In some embodiments, the radionuclide is an alpha emitter, e.g., Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), or Terbium-149 (¹⁴⁹Tb), or a progeny thereof. In some embodiments, the alpha-emitter is Actinium-225 (²²⁵Ac), or a progeny thereof.

### Linker

In some embodiments, the linker is as shown within the structure of Formula I-b, as that part of Formula I-b absent A and B:

A-L¹-(L²)ₙ-B Formula I-b

(A and B are as defined in Formula I-a.)

Thus, in some embodiments, the linker is -L¹-(L²)ₙ-, wherein:

L¹ is optionally substituted C₁-C₆ alkyl, optionally substituted C₁-C₆ heteroalkyl, or optionally substituted aryl or heteroaryl;

n is between 1 and 5 (inclusive); and

each L², independently, has the structure:

(—X¹-L³-Z¹—) Formula III

wherein:

X¹ is C═O(NR¹), C═S(NR¹), OC═O(NR¹), NR¹C═O(O), NR¹C═O(NR¹), —CH₂PhC═O(NR¹), —CH₂Ph(NH)C═S(NR¹), O, or NR¹; and each R¹ independently is H, optionally substituted C₁-C₆ alkyl, optionally substituted C₁-C₆ heteroalkyl, or optionally substituted aryl or heteroaryl, in which C₁-C₆ alkyl can be substituted by oxo (═O), heteroaryl, or a combination thereof;

L³ is optionally substituted C₁-C₅₀ alkyl or optionally substituted C₁-C₅₀ heteroalkyl (e.g., C₅-C₂₀ polyethylene glycol);

Z¹ is CH₂, C═O, C═S, OC═O, NR¹C═O, or NR¹, wherein R¹ is a hydrogen or optionally substituted C₁-C₆ alkyl or pyrrolidine-2,5-dione.

In some embodiments, L¹ is substituted C₁-C₆ alkyl or substituted C₁-C₆ heteroalkyl, the substituent comprising a heteroaryl group (e.g., six-membered nitrogen-containing heteroaryl).

In some embodiments, L³ is substituted C₁-C₅₀ alkyl or substituted C₁-C₅₀ heteroalkyl, the substituent comprising a heteroaryl group (e.g., six-membered nitrogen-containing heteroaryl).

In some embodiments, A is a macrocyclic chelating moiety comprising one or more heteroaryl groups (e.g., six-membered nitrogen-containing heteroaryl).

### Cross-Linking Groups

In some embodiments, radioimmunoconjugates comprise a cross-linking group instead of or in addition to the targeting moiety (e.g., B in Formula I comprises a cross-linking group).

A cross-linking group is a reactive group that is able to join two or more molecules by a covalent bond. Cross-linking groups may be used to attach the linker and chelating moiety to a therapeutic or targeting moiety. Cross-linking groups may also be used to attach the linker and chelating moiety to a target in vivo. In some embodiments, the cross-linking group is an amino-reactive, methionine reactive or thiol-reactive cross-linking group, or a sortase-mediated coupling. In some embodiments, the amino-reactive or thiol-reactive cross-linking group comprises an activated ester such as a hydroxysuccinimide ester, 2,3,5,6-tetrafluorophenol ester, 4-nitrophenol ester or an imidate, anhydride, thiol, disulfide, maleimide, azide, alkyne, strained alkyne, strained alkene, halogen, sulfonate, haloacetyl, amine, hydrazide, diazirine, phosphine, tetrazine, isothiocyanate, or oxaziridine. In some embodiments, the sortase recognition sequence may comprise of a terminal glycine-glycine-glycine (GGG) and/or LPTXG amino acid sequence, where X is any amino acid. A person having ordinary skill in the art will understand that the use of cross-linking groups is not limited to the specific constructs disclosed herein, but rather may include other known cross-linking groups.

### Pharmaceutical Compositions

In one aspect, the present disclosure provides pharmaceutical compositions comprising radioimmunoconjugates disclosed herein. Such pharmaceutical compositions can be formulated for use in a variety of drug delivery systems. One or more physiologically acceptable excipients or carriers can also be included in a pharmaceutical composition for proper formulation. Non-limiting examples of suitable formulations compatible for use with the present disclosure include those described in Remington's Pharmaceutical Sciences, Mack Publishing Company, Philadelphia, Pa., 17th ed., 1985. For a brief review of methods for drug delivery, See, e.g., Langer (Science. 249:1527-1533, 1990).

Pharmaceutical compositions may be formulated for any of a variety of routes of administration discussed herein (See, e.g., the “Administration and Dosage” subsection herein), Sustained release administration is contemplated, by such means as depot injections or erodible implants or components. Thus, the present disclosure provides pharmaceutical compositions that include agents disclosed herein (e.g., radioimmunoconjugates) dissolved or suspended in an acceptable carrier, preferably an aqueous carrier, e.g., water, buffered water, saline, or PBS, among others. In some embodiments, pharmaceutical compositions contain pharmaceutically acceptable auxiliary substances to approximate physiological conditions, such as pH adjusting and buffering agents, tonicity adjusting agents, wetting agents, or detergents, among others. In some embodiments, pharmaceutical compositions are formulated for oral delivery and may optionally contain inert ingredients such as binders or fillers for the formulation of a unit dosage form, such as a tablet or a capsule. In some embodiments, pharmaceutical compositions are formulated for local administration and may optionally contain inert ingredients such as solvents or emulsifiers for the formulation of a cream, an ointment, a gel, a paste, or an eye drop.

In some embodiments, provided pharmaceutical compositions are sterilized by conventional sterilization techniques, e.g., may be sterile filtered. Resulting aqueous solutions may be packaged for use as is, or lyophilized. Lyophilized preparations can be, for example, combined with a sterile aqueous carrier prior to administration. The pH of preparations typically will be between 3 and 11, more preferably between 5 and 9 or between 6 and 8, and most preferably between 6 and 7, such as 6 to 6.5. Resulting compositions in solid form may be packaged, for example, in multiple single dose units, each containing a fixed amount of the above-mentioned agent or agents, such as in a sealed package of tablets or capsules. Pharmaceutical compositions in solid form can also be packaged in a container for a flexible quantity, such as in a squeezable tube designed for a topically applicable cream or ointment.

### Methods of Treatment

In one aspect, the present disclosure provides methods of treatment comprising a subject a radioimmunoconjugate as disclosed herein.

### Subjects

In some disclosed methods, a therapy (e.g., comprising a therapeutic agent) is administered to a subject. In some embodiments, the subject is a mammal, e.g., a human.

In some embodiments, the subject has cancer or is at risk of developing cancer. For example, the subject may have been diagnosed with cancer. For example, the cancer may be a primary cancer or a metastatic cancer. Subjects may have any stage of cancer, e.g., stage I, stage II, stage III, or stage IV with or without lymph node involvement and with or without metastases. Provided radioimmunoconjugates and compositions may prevent or reduce further growth of the cancer and/or otherwise ameliorate the cancer (e.g., prevent or reduce metastases). In some embodiments, the subject does not have cancer but has been determined to be at risk of developing cancer, e.g., because of the presence of one or more risk factors such as environmental exposure, presence of one or more genetic mutations or variants, family history, etc. In some embodiments, the subject has not been diagnosed with cancer.

In some embodiments, the cancer is a solid tumor cancer, e.g., a sarcoma or carcinoma.

In some embodiments, the solid tumor cancer is adrenocortical carcinoma, bladder cancer (e.g., urothelial carcinoma), breast cancer, cervical cancer, colorectal cancer, endometrial adenocarcinoma, Ewing's sarcoma, gallbladder carcinoma, glioma (e.g., glioblastoma mutiforme), head and neck cancer, liver cancer, lung cancer (e.g., small cell lung cancer or non-small cell lung cancer, or adenocarcinoma of the lung), neuroblastoma, neuroendocrine cancer, pancreatic cancer (e.g., pancreatic exocrine carcinoma), prostate cancer, renal cell carcinoma, salivary adenoid cystic cancer, or spermatocytic seminoma.

In some embodiments, the cancer is selected from the group consisting of bladder cancer, breast cancer, head and neck cancer, liver cancer, and lung cancer. In some embodiments, the cancer is bladder cancer. In some embodiments, the cancer is head and neck cancer. In some embodiments, the cancer is liver cancer.

In some embodiments, the cancer is a non-solid tumor cancer, e.g., a liquid cancer or hematologic cancer. In some embodiments, the cancer is a myeloma, e.g., multiple myeloma. In some embodiments, the cancer is a leukemia, e.g., acute myeloid leukemia. In some embodiments, the cancer is a lymphoma.

### Administration and Dosage

Radioimmunoconjugates and pharmaceutical compositions thereof disclosed herein may be administered by any of a variety of routes of administration, including systemic and local routes of administration

Systemic routes of administration include parenteral routes and enteral routes. In some embodiments, radioimmunoconjugates or pharmaceutical compositions thereof are administered by a parenteral route, for example, intravenously, intraarterially, intraperitoneally, subcutaneously, or intradermally. In some embodiments, radioimmunoconjugates or pharmaceutical compositions thereof are administered intravenously. In some embodiments, radioimmunoconjugates or pharmaceutical compositions thereof are administered by an enteral route of administration, for example, trans-gastrointestinal, or orally.

Local routes of administration include, but are not limited to, peritumoral injections and intratumoral injections.

Pharmaceutical compositions can be administered for radiation treatment planning, diagnostic, and/or therapeutic treatments. When administered for radiation treatment planning or diagnostic purposes, the radioimmunoconjugate may be administered to a subject in a diagnostically effective dose and/or an amount effective to determine the therapeutically effective dose. In therapeutic applications, pharmaceutical compositions may be administered to a subject (e.g., a human) already suffering from a condition (e.g., cancer) in an amount sufficient to cure or at least partially arrest the symptoms of the disorder and its complications. An amount adequate to accomplish this purpose is defined as a “therapeutically effective amount,” an amount of a compound sufficient to substantially improve at least one symptom associated with the disease or a medical condition. For example, in the treatment of cancer, an agent or compound that decreases, prevents, delays, suppresses, or arrests any symptom of the disease or condition would be therapeutically effective. A therapeutically effective amount of an agent or compound is not required to cure a disease or condition but may, for example, provide a treatment for a disease or condition such that the onset of the disease or condition is delayed, hindered, or prevented, such that the disease or condition symptoms are ameliorated, or such that the term of the disease or condition is changed. For example, the disease or condition may become less severe and/or recovery is accelerated in an individual. In some embodiments, a subject is administered a first dose of a radioimmunoconjugate or composition in an amount effective for radiation treatment planning, then administered a second dose or set of doses of the radioimmunoconjugate or composition in a therapeutically effective amount.

Effective amounts may depend on the severity of the disease or condition and other characteristics of the subject (e.g., weight). Therapeutically effective amounts of disclosed radioimmunoconjugates and compositions for subjects (e.g., mammals such as humans) can be determined by the ordinarily-skilled artisan with consideration of individual differences (e.g., differences in age, weight, and the condition of the subject.

In some embodiments, disclosed radioimmunoconjugates exhibit an enhanced ability to target cancer cells. In some embodiments, effective amount of disclosed radioimmunoconjugates are lower than (e.g., less than or equal to about 90%, 75%, 50%, 40%, 30%, 20%, 15%, 12%, 10%, 8%, 7%, 6%, 5%, 4%, 3%, 2%, 1%, 0.5%, or 0.1% of) the equivalent dose for a therapeutic effect of the unconjugated, and/or non-radiolabeled targeting moiety.

Single or multiple administrations of pharmaceutical compositions disclosed herein including an effective amount can be carried out with dose levels and pattern being selected by the treating physician. Dose and administration schedule can be determined and adjusted based on the severity of the disease or condition in the subject, which may be monitored throughout the course of treatment according to the methods commonly practiced by clinicians or those described herein.

The following specific examples are to be construed as merely illustrative, and not limitative of the remainder of the disclosure in any way whatsoever.

### EXAMPLES

### Example 1. General Materials and Methods

Lutetium-177 can be obtained from Perkin Elmer as lutetium trichloride in a 0.05 N hydrochloric acid solution; indium-111, as a trichloride salt, can be obtained from Nordion; and actinium-225 can be obtained as actinium-225 trinitrate from Oak Ridge National Laboratories.

Analytical HPLC-MS can be performed using a Waters Acquity HPLC-MS system comprised of a Waters Acquity Binary Solvent Manager, a Waters Acquity Sample Manager (samples cooled to 10° C.), a Water Acquity Column Manager (column temperature 30° C.), a Waters Acquity Photodiode Array Detector (monitoring at 254 nm and 214 nm), a Waters Acquity TQD with electrospray ionization and a Waters Acquity BEH C18, 2.1×50 (1.7 μm) column. Preparative HPLC can be performed using a Waters HPLC system comprised of a Waters 1525 Binary HPLC pump, a Waters 2489 UV/Visible Detector (monitoring at 254 nm and 214 nm) and a Waters XBridge Prep phenyl or C18 19×100 mm (5 μm) column.

HPLC elution method 1: Waters Acquity BEH C18 2.1×50 mm (1.7 μm) column; mobile phase A: H₂O (0.1% v/v TFA); mobile phase B: acetonitrile (0.1% v/v TFA); flow rate=0.3 mL/min; initial=90% A, 3-3.5 min=0% A, 4 min=90% A, 5 min=90% A.

HPLC elution method 2: Waters XBridge Prep Phenyl 19×100 mm (5 μm) column; mobile phase A: H₂O (0.1% v/v TFA); mobile phase B: acetonitrile (0.1% v/v TFA); flow rate: 10 mL/min; initial=80% A, 13 min=0% A.

HPLC elution method 3: Waters Acquity BEH C18 2.1×50 mm (1.7 μm) column; mobile phase A: H₂O (0.1% v/v TFA); mobile phase B: acetonitrile (0.1% v/v TFA); flow rate=0.3 mL/min; initial=90% A, 8 min=0% A, 10 min=0% A, 11 min=90% A, 12 min=90% A.

HPLC elution method 4: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=80% A, 3 min=80% A, 13 min=20% A, 18 min=0% A.

HPLC elution method 5: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=90% A, 3 min=90% A, 13 min=0% A, 20 min=0% A.

HPLC elution method 6: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=75% A, 13 min=0% A, 15 min=0% A.

HPLC elution method 7: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=80% A, 12 min=0% A, 15 min=0% A.

HPLC elution method 8: Waters XBridge Prep C18 OBD 19×100 mm (5 μm) column; mobile phase A: H₂O (0.1% v/v TFA); mobile phase B: acetonitrile (0.1% v/v TFA); flow rate: 10 mL/min; initial=90% A, 12 min=0% A, 15 min=0% A.

Analytical Size Exclusion Chromatography (SEC) can be performed using a Waters system comprised of a Waters 1525 Binary HPLC pump, a Waters 2489 UV/Visible Detector (monitoring at 280 nm), a Bioscan Flow Count radiodetector (FC-3300) and TOSOH TSKgel G3000SWxl, 7.8×300 mm column. The isocratic SEC method can have a flow rate of, e.g., mL/min, with a mobile phase of 0.1 M phosphate, 0.6 M NaCl, 0.025% sodium azide, pH=7.

MALDI-MS (positive ion) can be performed using a MALDI Bruker Ultraflextreme Spectrometer.

Radio thin-layer chromatography (radioTLC) can be performed with Bioscan AR-2000 Imaging Scanner, and can be carried out on iTLC-SG glass microfiber chromatography paper (Agilent Technologies, SGI0001) plates using citrate buffer (0.1 M, pH 5.5).

### Example 2. Synthesis of 4-{[11-oxo-11-(2,3,5,6-tetrafluorophenoxy)undecyl]carbamoyl}-2-[4,7,10-tris(carboxymethyl)-1,4,7,10-tetraazacyclododecan-1-yl]butanoic acid (Compound B)

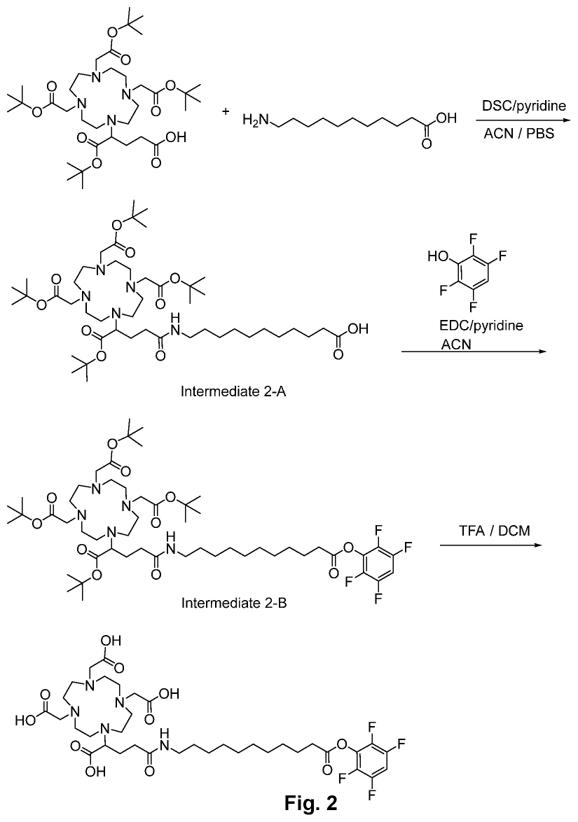

A bifunctional chelate, 4-{[11-oxo-11-(2,3,5,6-tetrafluorophenoxy)undecyl]carbamoyl}-2-[4,7,10-tris(carboxymethyl)-1,4,7,10-tetraazacyclododecan-1-yl]butanoic acid (Compound B), can be synthesized according to the scheme provided in FIG. 2. To a solution of 5-(tert-butoxy)-5-oxo-4-(4,7,10-tris(2-(tert-butoxy)-2-oxoethyl)-1,4,7,10-tetraazacyclododecan-1-yl)pentanoic acid (DOTA-GA-(tBu)₄, 50 mg, 0.07 mmol) in ACN (2.0 mL), DSC (50 mg, 0.21 mmol) is added, followed by pyridine, (0.20 mL, 2.48 mmol). The reaction is stirred at room temperature for 1 hour. To the reaction mixture is added 11-aminoundecanoic acid, (70 mg, 0.36 mmol) followed by PBS solution (1.0 mL) at room temperature. The reaction is stirred for 72 hours at room temperature. The reaction mixture is filtered with a syringe filter and purified directly by Prep-HPLC using method 6 to yield Intermediate 2-A.

To a solution of Intermediate 2-A (40 mg, 0.03 mmol), TFP (90 mg, 0.54 mmol) and EDC (40 mg, 0.27 mmol) in ACN (1.0 mL) is added pyridine (0.05 mL, 50 mg, 0.62 mmol) at room temperature. The solution is stirred at room temperature for 24 hours. The reaction is purified directly by Prep-HPLC using method 7 to provide Intermediate 2-B as a wax after concentration using a Biotage V10 Rapid Evaporator.

Intermediate 2-B is dissolved in DCM/TFA (1.0 mL/2.0 mL) and allowed to stir at room temperature for 24 hours. The reaction is concentrated by air stream and purified directly by Prep-HPLC using method 8 to yield Compound B as a clear wax after concentration. An aliquot is analyzed by HPLC-MS elution method 3.

¹H NMR (600 MHz, DMSO-d₆) δ 7.99-7.88 (m, 1H), 7.82 (t, J=5.5 Hz, 1H), 3.78 (broad s, 4H), 3.43 (broad s, 12H), 3.08 (broad s, 4H), 3.00 (m, 3H), 2.93 (broad s, 3H), 2.77 (t, J=7.2 Hz, 2H), 2.30 (broad s, 2H), 1.88 (broad s, 2H), 1.66 (p, J=7.3 Hz, 2H), 1.36 (m, 4H), 1.32-1.20 (m, 9H).

### Example 3. Synthesis of [²²⁵Ac]-Compound B-Anti-FGFR3 Conjugate

Compound B (1 μmole) is dissolved in a hydrochloric acid solution (0.001 M). An aliquot of Compound B solution (5 μL, 70 nmole) is added to a solution containing an anti-FGFR3 antibody (1.8 nmoles) in a phosphate buffer (pH 8). After 3 hours at ambient temperature, the resulting immunoconjugate is purified via a Sephadex G-50 resin packed column. The immunoconjugate Compound B-anti-FGFR3 is eluted from the column with acetate buffer (pH 6.5).

Ac-225 (15 μCi, 10 μL) is added to a solution of Compound B-anti-FGFR3 (300 μg in acetate buffer (pH 6.5). The radiolabeling reaction is incubated at 30° C. for 1 hour. The crude product, [²²⁵Ac]-Compound B-anti-FGFR3, is purified via a Sephadex G-50 resin packed column eluted with acetate buffer.

### Example 4. Synthesis of 4-{[2-(2-{2-[3-oxo-3-(2,3,5,6-tetrafluorophenoxy)propoxy]ethoxy}ethoxy)ethyl]carbamoyl}-2-[4,7,10-tris(carboxymethyl)-1,4,7,10-tetraazacyclododecan-1-yl]butanoic acid (Compound C)

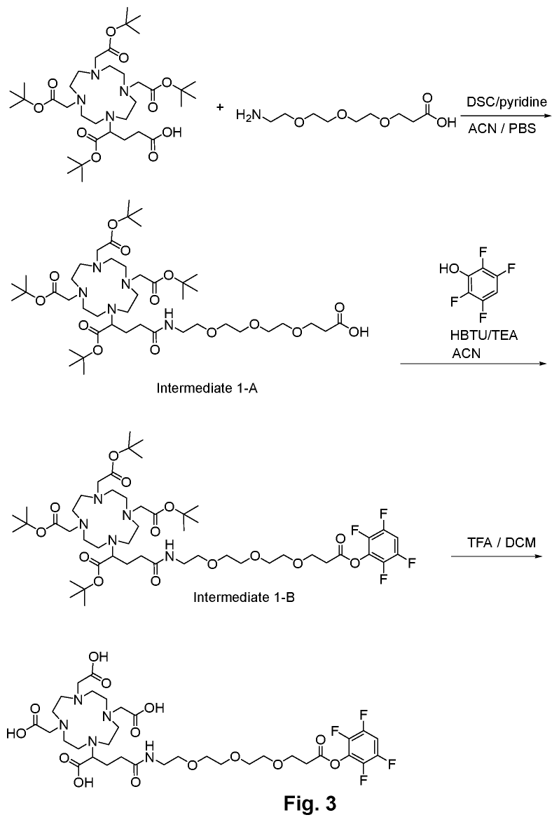

A bifunctional chelate, 4-{[2-(2-{2-[3-oxo-3-(2,3,5,6-tetrafluorophenoxy)propoxy] ethoxy}ethoxy)ethyl]carbamoyl}-2-[4,7,10-tris(carboxymethyl)-1,4,7,10-tetraazacyclododecan-1-yl]butanoic acid (Compound C), is synthesized according to the scheme provided in FIG. 3.

To a solution of 5-(tert-butoxy)-5-oxo-4-(4,7,10-tris(2-(tert-butoxy)-2-oxoethyl)-1,4,7,10-tetraazacyclododecan-1-yl)pentanoic acid (DOTA-GA(tBu)₄, 100 mg, 0.143 mmol) in ACN (8.0 mL) is added DSC (73 mg, 0.285 mmol) and pyridine (0.80 mL, 9.89 mmol). The reaction mixture is stirred for 90 min at ambient temperature. This solution is added to a semi-solution of amino-PEG3-acid (63 mg, 0.285 mmol in 1.2 mL of DMF) in a 100 mL round bottom flask. After 4 hours at ambient temperature, the reaction is worked up by concentrating to dryness under a stream of air. The crude material is purified by HPLC elution method 2 (dissolved the crude in 6 mL of 20% ACN/H₂O). The fractions containing product are pooled and concentrated under vacuum and then co-evaporated with ACN (3×2 mL).

To a vial containing Intermediate 1-A (82 mg, 60 μmol) is added ACN (2 mL), NEt₃ (50 μL, 360 μmol, 6 equiv.), HBTU (23 mg, 60 μmol, 1 equiv) and a TFP solution (50 mg, 300 μmol, 5 equiv., dissolved in 250 μL of ACN). The resulting clear solution is stirred at ambient temperature for 3 hours. The reaction is worked up by concentrating the solution to dryness under an air stream and is then diluted with ACN/H₂O (1:1, 3 mL total) and purified on preparative HPLC using elution method 4. Fractions containing product are pooled and concentrated under vacuum and then co-evaporated with ACN (3×2 mL). Intermediate 1-B is obtained as a clear residue.

To a vial containing Intermediate 1-B (67 mg, 64 μmol) is added DCM (2 mL) and TFA (2 mL). The resulting solution is stirred at ambient temperature for 16 hour. Additional, TFA (2 mL) is added, and the reaction is stirred at ambient temperature for 6 hours. The reaction is concentrated to dryness under an air stream, with the crude product being finally dissolved in ACN/H₂O (1 mL of 10% ACN/H₂O). The crude reaction solution isthen purified by preparative HPLC using elution method 5. The fractions containing product are pooled, frozen and lyophilized. Compound C is obtained as a white solid. An aliquot is analyzed by HPLC-MS elution method 3.

¹H NMR (DMSO-d₆, 600 MHz) δ 7.97-7.91 (m, 2H), 3.77 (t, 2H, J=6.0 Hz), 3.58-3.55 (m, 2H), 3.53-3.48 (m, 8H), 3.44-3.38 (m, 10H), 3.23-3.08 (m, 11H), 3.02 (t, 2H, J=6.0 Hz), 2.93 (broad s, 4H), 2.30 (broad s, 2H), 1.87 (broad s, 2H).

### Example 5. Synthesis of [²²⁵Ac]-Compound C-Anti-FGFR3 Conjugate

Compound C (1 μmole) is dissolved in a hydrochloric acid solution (0.001 M). An aliquot of Compound C solution (5 μL, 70 nmole) is added to a solution containing anti-FGFR3 antibody (1.8 nmoles) in a phosphate buffer (pH 8). After 3 hours at ambient temperature, the resulting immunoconjugate is purified via a Sephadex G-50 resin packed column. The immunoconjugate Compound C-anti-FGFR3 is eluted from the column with acetate buffer (pH 6.5). Identities of eluates can be confirmed by, e.g., MALDI-TOF.

Ac-225 (15 μCi, 10 μL) is added to a solution of Compound C-anti-FGFR3 (300 μg in acetate buffer (pH 6.5). The radiolabeling reaction is incubated at 30° C. for 1 hour. The crude product, [²²⁵Ac]-Compound C-anti-FGFR3, is purified via a Sephadex G-50 resin packed column eluted with acetate buffer.

### Example 6. Effects of [²²⁵Ac]-Anti-FGFR3 Conjugates on Tumor Growth and Survival in a Bladder Cancer Xenograft Model

[²²⁵Ac]-anti-FGFR3 conjugates are tested using the human UM-UC-1 bladder cell line, which expresses wild type FGFR3. UM-UC-1 cells are injected into immunocompromised mice. After the establishment of tumors, mice are administered an [²²⁵Ac]-anti-FGFR3 conjugate, control (e.g., PBS buffer or other vehicle alone), or optionally unconjugated anti-FGFR3.

Tumor volume is monitored twice weekly using caliper measurements, and the results are compared across treatment groups. Survival is recorded. Greater inhibition of tumor growth and/or greater survival in [²²⁵Ac]-anti-FGFR3 conjugate treatment groups indicates increased efficacy.

### Example 7. Effects of [²²⁵Ac]-Anti-FGFR3 Conjugates on Tumor Growth and Survival in WT and Mutant FGFR3 Bladder Cancer Xenograft Models

While wild-type FGFR3 is overexpressed in certain cancers, some tumors are associated with mutant FGFR3. In this Example, [²²⁵Ac]-anti-FGFR3 conjugates are tested using various human bladder cell lines that express either wild type or mutant FGFR3.

RT112 bladder cancer cells, which express WT FGFR3, are injected into nude (nu/nu) mice, and tumors are allowed to grow to a mean volume of ˜100-150 mm³. Animals are dosed twice weekly with vehicle or with an [²²⁵Ac]-anti-FGFR3 conjugate. Optionally, a third set of animals are dosed with unconjugated anti-FGFR3.

Tumors are measured twice weekly using a caliper, and tumor volume is calculated using the formula:

V=0.5×a×b²

wherein a and b are the length and width of the tumor, respectively.

Tumor growth is compared across groups.

To assess the effects of [²²⁵Ac]-anti-FGFR3 conjugates on FGFR3 signaling, tumor lysates are collected at 48 and 72 hour after treatment. Phosphorylation and total protein levels of FRS2α, AKT, and p44/42 MAPK (downstream mediators of FGFR3 signaling) in tumor lysates are examined.

Additionally, effects of [²²⁵Ac]-anti-FGFR3 conjugates are studied in a Ba/F3-FGFR3^{S249C} allograft model. See, e.g., Qing et al., “Antibody-based targeting of FGFR3 in bladder carcinoma and t(4;14)-positive multiple myeloma in mice.” J Clin Invest. 2009 May 1; 119(5): 1216-1229. (S249C is the most frequent FGFR3 mutation found in bladder cancer.) Tumor growth and tumor lysates are assessed as mentioned above for the RT112 xenograft model.

### Example 8. Effects of [²²⁵Ac]-Anti-FGFR3 Conjugates on Tumor Growth and Survival in Multiple Myeloma Xenograft Models

OPM2 and KMS11 are t(4:14)+ multiple myeloma cell lines harboring K650E and Y373C FGFR3 mutations, respectively. [²²⁵Ac]-anti-FGFR3 conjugates are tested in OPM2 and KMS11 xenograft models. Cells are expanded, and 15×10⁶ OPM2 or 20×10⁶ KMS11 cells are implanted subcutaneously into the flanks of mice in a volume of 0.2 ml in Hank's Balanced Salt Solution (HBSS)/Matrigel (1:1 v/v: BD Biosciences). Tumors are measured twice weekly as a caliper, and tumor volume is calculated as described in Example 7.

When tumors reach an average size of 150-200 mm³, animals are randomly assigned to a treatment or control group. Each [²²⁵Ac]-anti-FGFR3 conjugate may be tested in a separate treatment group. A control group may include mice administered HBSS or other vehicle. Optionally, for comparison, one or more treatment groups are included in which mice are administered unconjugated anti-FGFR3 (cold antibody). Mice in all groups are administered the relevant agents for their group twice weekly intraperitoneally.

Tumor volume is monitored twice weekly using caliper measurements, and the results are compared across treatment groups. Survival is recorded. Greater inhibition of tumor growth and/or greater survival in [²²⁵Ac]-anti-FGFR3 conjugate treatment groups indicates increased efficacy.

### Example 9. Effects of [²²⁵Ac]-Anti-FGFR3 Conjugates on Tumor Growth and Survival in a Liver Cancer Xenograft Model

[²²⁵Ac]-anti-FGFR3 conjugates are tested in a tumor xenograft model based on a liver cancer cell line (Huh7) essentially as described in Example 8.

### Example 10. Effects of [²²⁵Ac]-Anti-FGFR3 Conjugates on Tumor Growth and Survival in a Breast Cancer Xenograft Model

[²²⁵Ac]-anti-FGFR3 conjugates are tested in a tumor xenograft model based on a breast cancer cell line (Cal-51) essentially as described in Example 8.

### Example 11. Effects of [²²⁵Ac]-Anti-FGFR3 Conjugates on Tumor Growth and Survival in a Colon Adenocarcinoma Xenograft Model

[²²⁵Ac]-anti-FGFR3 conjugates are tested in the MC38 mouse colon adenocarcinoma xenograft model. FGFR3-positive MC38 cells are expanded, and 1×10⁶ MC38 cells are implanted subcutaneously into the flanks of female C₅₇BL/6 mice that are 8 to 12 weeks of age. When tumors reach an average size of 80-120 mm³, animals are pair matched and assigned to a treatment or control group. Each [²²⁵Ac]-anti-FGFR3 conjugate may be tested in a separate treatment group. A control group may include mice administered phosphate-buffered saline (PBS). Optionally, for comparison, one or more treatment groups are included in which mice are administered unconjugated anti-FGFR3 (cold antibody). Mice in all groups may be administered the relevant agents for their group according to a regular schedule, e.g., weekly, twice a week, or thrice per week, for one or more (e.g., 1, 2, or 3) weeks intravenously or intraperitoneally.

Tumor volume is monitored twice weekly using caliper measurements, and the results are compared across treatment groups. Survival is recorded. Greater inhibition of tumor growth and/or greater survival in [²²⁵Ac]-anti-FGFR3 conjugate treatment groups indicates increased efficacy.

### Example 12. Effects of [²²⁵Ac]-Anti-FGFR3 Conjugates on Immune Cell Infiltration Using an Adenocarcinoma Cell Line

MC38 (adenocarcinoma) cells are implanted subcutaneously into the flanks of female C57BL/6 mice that are 8 to 12 weeks of age. When tumors reach an average size of 80-120 mm³, animals are pair matched and divided into treatment and control group. A control group of mice receive PBS, immunoconjugate treatment group(s) receive [²²⁵Ac]-anti-FGFR3 conjugates, and optional antibody treatment group(s) receive unconjugated anti-FGFR3. All groups are administered according to the same route and dosing schedule: twice weekly intravenously.

After 7 days of treatment, half of the animals from each group are sacrificed, and tumors are collected. After 14 days of treatment, the remaining half of animals in each group are sacrificed, and tumors are collected. Half of each tumor is processed for paraffin embedding, while the other half is used to prepare a single cell suspension for flow cytometry analyses. Samples for flow cytometry analyses are stained for CD8 and for markers of T-regulatory cells. Higher ratios of CD8+ to regulatory T cells may indicate enhanced efficacy via immune cell infiltration into tumors.

### Example 13. Effects of [²²⁵Ac]-Anti-FGFR3 Conjugates on Lung Tumor Development

[²²⁵Ac]-anti-FGFR3 conjugates are tested in two mouse lung cancer xenograft models: Madison 109 (M109) and Lewis Lung Carcinoma cells, both of which are FGFR3-positive. 1×10⁶ Lewis Lung Carcinoma tumor cells are implanted subcutaneously into flanks of female C57BL/6 mice that are 8 to 12 weeks of age. Additionally, 1×10⁶ Madison 109 tumor cells are implanted subcutaneously into the flanks of CR female BALB/c mice that are 8 to 12 weeks of age.

When tumors reach an average size of 100-200 mm³, animals are pair matched and treatment is initiated. Each [²²⁵Ac]-anti-FGFR3 conjugate may be tested in a separate treatment group. A control group may include mice administered phosphate-buffered saline (PBS). Optionally, for comparison, one or more treatment groups are included in which mice are administered unconjugated anti-FGFR3 (cold antibody). Mice in all groups may be administered (intravenously or intraperitoneally) the relevant agents for their group according to a regular schedule, e.g., weekly, twice a week, or thrice per week. In this example, mice are treated for one, two, or three weeks (see below).

Tumors are measured using calipers twice weekly, and the results are compared across treatment groups. Greater inhibition of tumor growth in [²²⁵Ac]-anti-FGFR3 conjugate treatment groups indicates increased efficacy.

After 7 days of treatment, some of the animals from each group are sacrificed, and tumors are collected. After 14 days of treatment, some of the remaining animals in each group are sacrificed, and tumors are collected. The remaining animals continue to be dosed until day 21, at which time they are sacrificed and their tumors are collected. Half of each tumor is processed for paraffin embedding, while the other half is frozen in Optimal Cutting Temperature (O.C.T.) compound.

### Example 14. Effects of [²²⁵Ac]-Anti-FGFR3 Conjugates on Survival

Example 12 and/or 13 is performed, except that mice are not sacrificed and are instead monitored for tumor growth and survival over a period of at least months. Enhanced survival in [²²⁵Ac]-anti-FGFR3 conjugate treatment groups indicates enhanced therapeutic efficacy.

### Example 15. Effects of [²²⁵Ac]-Anti-FGFR3 Conjugates on Tumor Growth and Survival in a Bladder Cancer Cell Lines Involving FGFR3 Fusions

[²²⁵Ac]-anti-FGFR3 conjugates are tested in a tumor xenograft models based on one or more of the RT4, RT112, SW780, and UMUC-14 bladder cell lines, essentially as described in Example 8. RT4 and RT112 cells contain an FGFR3-TACC3 fusion, SW780 cells contain an FGFR-BAIAP2L1 fusion, and UMUC-14 harbors an FGFR3^{S249C}.

### Example 16. Binding of DOTA-Anti-FGFR3 Conjugate to Cancer Cells Expressing FGFR3

The present Example demonstrates binding of conjugated anti-FGFR3 to FGFR3-positive cancer cells at subnanomolar/picomolar Kd ranges.

An unlabeled DOTA-anti-FGFR3 conjugate was synthesized using 1) a pure R enantiomer of Compound C (see Example 4) (that is, an R-enantiomer of a (2R)-2-[4,7,10-tris(carboxymethyl)-1,4,7,10-tetraazacyclododecan-1-yl]pentanedioic acid (R-DOTA-GA), connected through a PEG3 acid linker to a 2,3,5,6-tetrafluorophenol active ester) and 2) MFGR1877S (vofatamab), an anti-FGFR3 antibody. Binding of DOTA-anti-FGFR3 to FGFR3-positive cancer cell lines RT4 (bladder), RT112 (bladder), and HepG2 (liver) was assessed by flow cytometry.

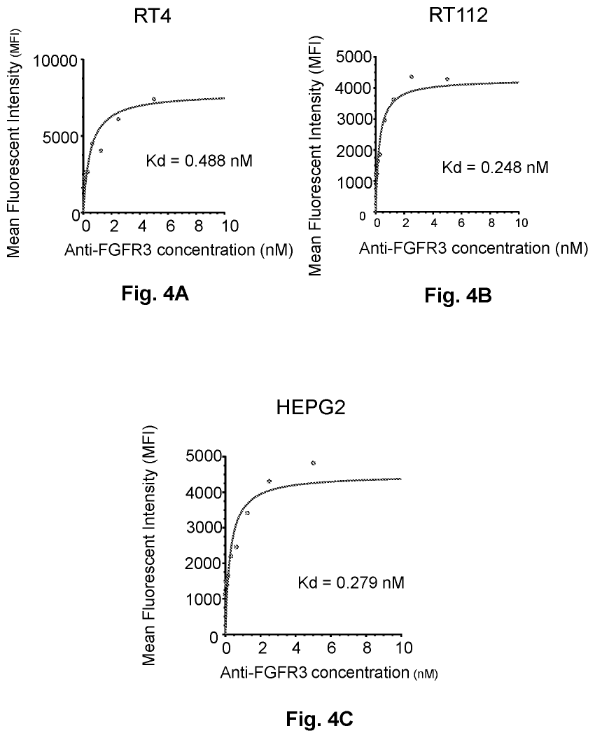

FIGS. 4A, 4B, and 4C show the binding curves for RT4, RT112, and HepG2, respectively, and the corresponding binding affinities (Kd) are summarized in Table 2.

TABLE 2
Binding affinities of anti-FGFR3 conjugate to FGFR3+ cancer cells

| | Kd [nM] | Kd [nM] | Kd [nM] |
|---|---|---|---|
| | RT4 | RT112 | HepG2 |
| Anti-FGFR3 conjugate | 0.448 | 0.248 | 0.279 |

### Example 17. In Vivo Biodistribution of [¹⁷⁷Lu]-DOTA-Anti-FGFR3 Conjugate

A Balb/c nude/RT4 cell line xenograft mouse model was used to assess the in vivo biodistribution of a radiolabeled anti-FGFR3 conjugate. A [¹⁷⁷Lu]-DOTA-anti-FGFR3 conjugate was synthesized using a pure R enantiomer of Compound C (see Example 4), MFGR1877S (vofatamab), and lutetium-177.

Groups of tumor-bearing animals were injected intravenously with [¹⁷⁷Lu]-DOTA-anti-FGFR3. Doses contained about 23 microcuries (μCi) of activity on 2 μg (0.1 mg/kg) of antibody. Animals were euthanized at 4 h, 24 h, 48 h, 96 h, and 168 h after injection to determine levels of radioactivity in the blood, kidney, liver, lungs, spleen, skin, tumor, and tail (n=3 per time point).

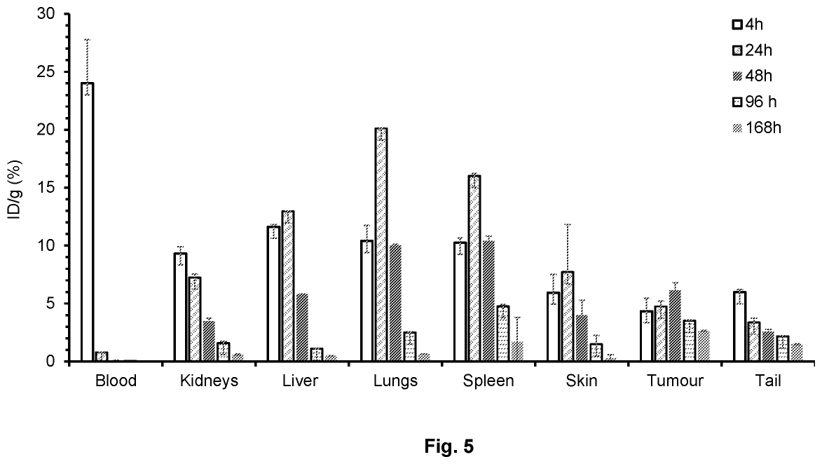

Results were expressed as the percentage injected dose per gram of tissue (% ID/g) and are depicted in FIG. 5. [¹⁷⁷Lu]-DOTA-anti-FGFR3 cleared rapidly from the blood and demonstrated transient uptake in the liver, lungs, and spleen. Tumor uptake was about 5% ID/g at all time points. Without wishing to be bound by any particular theory, the observed level of tumor uptake could be attributable to the small size of the RT4 tumors (about 50 mm³).

### Example 18. In Vivo Biodistribution of [¹⁷⁷Lu]-DOTA-Anti-FGFR3 Conjugate after Pre-Dosing with Cold Anti-FGFR3

The present Example demonstrates uptake of presently disclosed radioimmunoconjugates in tumor cells with lower levels of uptake in normal tissues.

A Balb/c nude/RT112 cell line xenograft mouse model was used to assess the in vivo biodistribution of [¹⁷⁷Lu]-DOTA-anti-FGFR3 after pre-dosing with cold (non-radiolabeled, unconjugated) anti-FGFR3 antibody.

Groups of tumor-bearing mice were injected intravenously with [¹⁷⁷Lu]-DOTA-anti-FGFR3. Doses contained about 23 microcuries (μCi) of activity on 2 μg (0.1 mg/kg) of antibody. Approximately three hours before administration of [¹⁷⁷Lu]-DOTA-anti-FGFR3, half of the mice were administered 100 μg cold anti-FGFR3 (vofatamab) by intraperitoneal injection. Animals were euthanized at 4 h, 24 h, 48 h, and 96 h after injection to determine levels of radioactivity in the blood, intestine (small and large), kidney and adrenal glands, liver and gall bladder, lungs, spleen, skin, bladder, urine, and tumor (n=3 per time point).

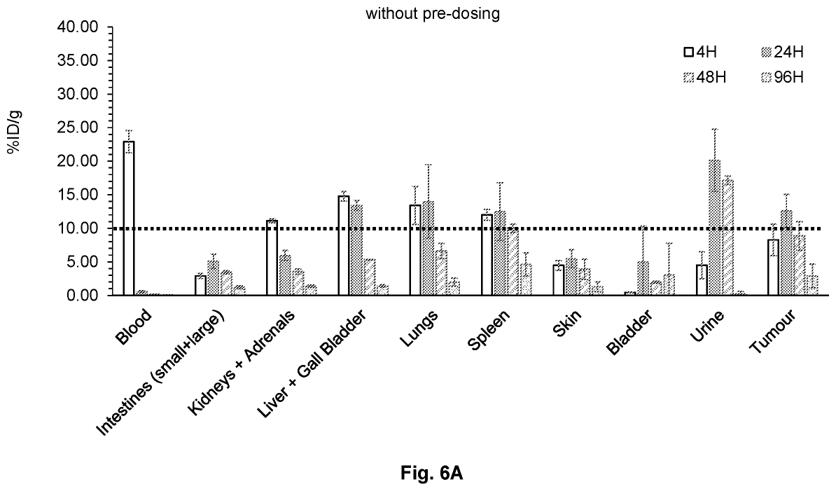

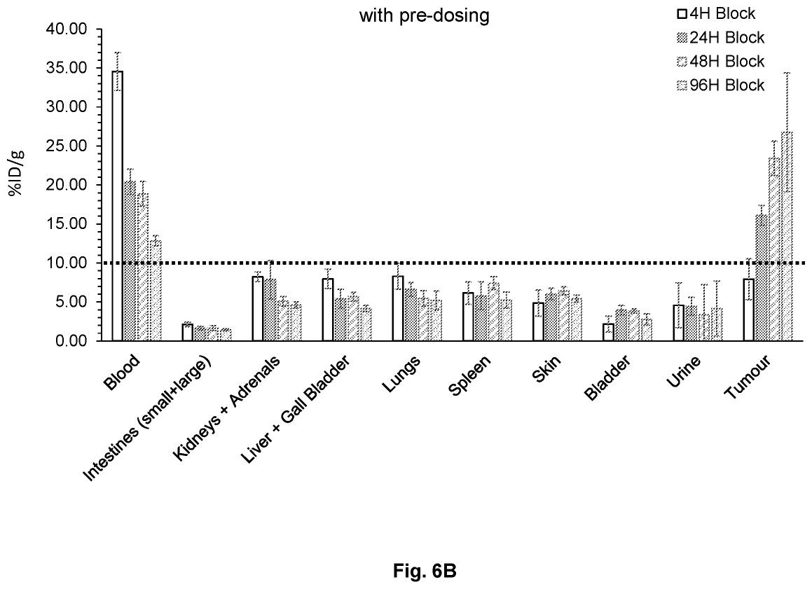

Results were expressed as the % ID/g and are depicted in FIGS. 6A and 6B. Pre-dosing with cold anti-FGFR3 reduced clearance of radioactivity from the blood, reduced uptake of [¹⁷⁷Lu]-DOTA-anti-FGFR3 in normal tissues, and increased uptake of [¹⁷⁷Lu]-DOTA-anti-FGFR3 in tumors.

### Example 19. In Vivo Biodistribution of Radiolabeled Anti-FGFR3 Conjugates Co-Dosed with Cold Anti-FGFR3

The present Example demonstrates uptake of presently disclosed radioimmunoconjugates in tumor cells with lower levels of uptake in normal tissues. Moreover, the present Example demonstrates that DOTA-anti-FGFR3 conjugates labeled with different radionuclides exhibit similar biodistribution profiles.

[¹¹¹In]-DOTA-anti-FGFR3 conjugate was synthesized using a pure R enantiomer of Compound C (see Example 4), MFGR1877S (vofatamab), and indium-111.

A Balb/c nude/RT112 cell line xenograft mouse model was used to assess the in vivo biodistribution of [¹⁷⁷Lu]-DOTA-anti-FGFR3 conjugate and [¹¹¹In]-DOTA-anti-FGFR3 conjugates when co-dosed with cold anti-FGFR3.

Groups of tumor-bearing mice were injected intravenously with [¹⁷⁷Lu]-DOTA-anti-FGFR3 at about 22 microcuries (μCi) of activity on 2 μg (0.1 mg/kg) of antibody. Mice were also co-administered 50, 100, or 200 μg of cold anti-FGFR3 via the same intravenous injection. Animals were euthanized at 24 h and 96 h after injection to determine levels of radioactivity in the blood, intestine, kidney, liver, lungs, spleen, skin, bladder, urine, and tumor (n=3 per time point).

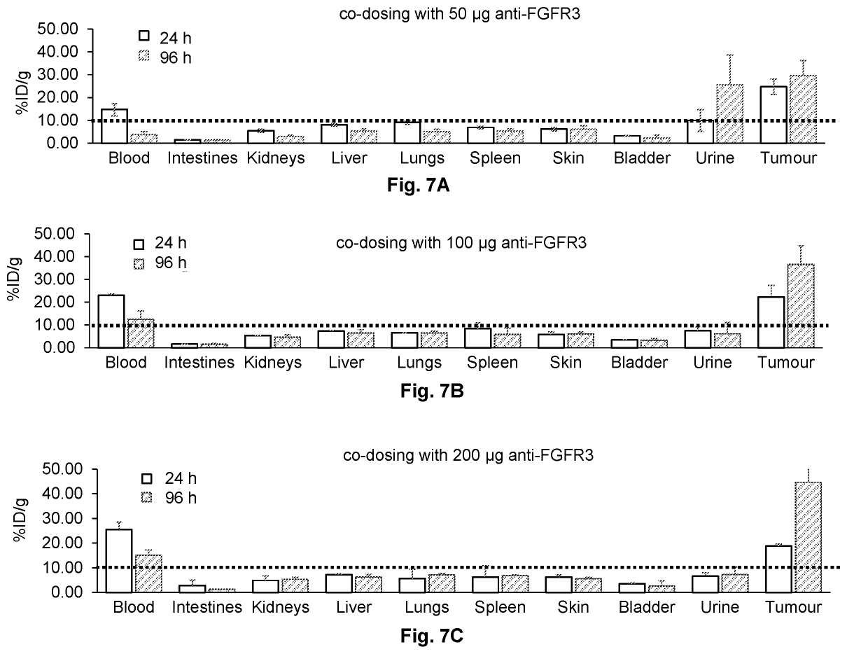

Results were expressed as the % ID/g and depicted in FIGS. 7A-7C. Co-dosing with 100 μg or 200 μg cold anti-FGFR3 reduced clearance of radioactivity from the blood, reduced uptake of [¹⁷⁷Lu]-DOTA-anti-FGFR3 in normal tissues, and increased uptake of [¹⁷⁷Lu]-DOTA-anti-FGFR3 in tumors.

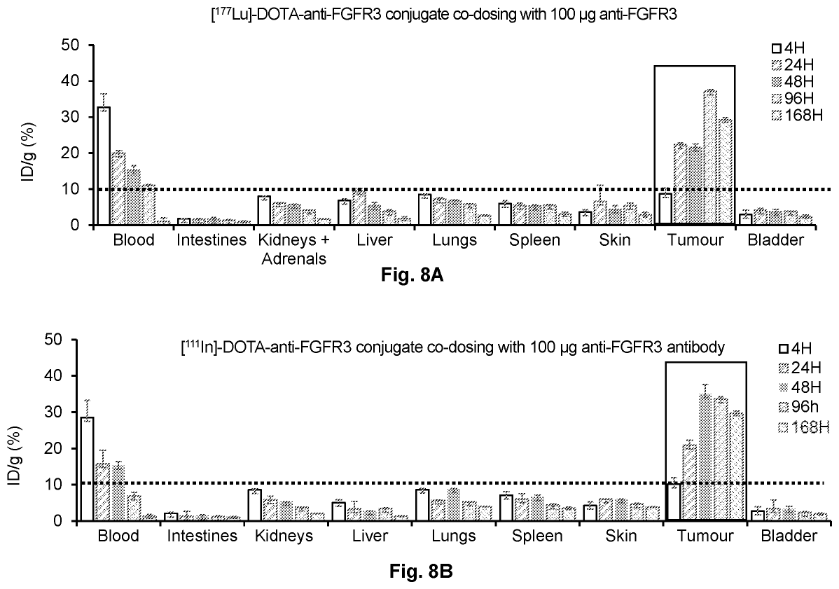

A biodistribution study was also performed using [¹¹¹In]-DOTA-anti-FGFR3 co-dosed with 100 μg of cold anti-FGFR3, similarly as described for the [¹⁷⁷Lu]-DOTA-anti-FGFR3 co-dosing experiment in this Example. FIGS. 8A and 8B show the results % ID/g in mice administered [¹⁷⁷Lu]-DOTA-anti-FGFR3 (FIG. 8A) or [¹⁷⁷Lu]-DOTA-anti-FGFR3 (FIG. 8B), each co-dosed with cold anti-FGFR3. Both [¹⁷⁷Lu]-DOTA-anti-FGFR3 and [¹¹¹In]-DOTA-anti-FGFR3 showed good tumor intake with about 34%-37% ID/g at 96 h after dosing.

### Example 20. Effects of [²²⁵Ac]-DOTA-Anti-FGFR3 Conjugate on Tumor Growth and Survival in a Bladder Cancer Xenograft Model

The present Example demonstrates therapeutic efficacy of an [²²⁵Ac]-DOTA-anti-FGFR3 conjugate in a bladder cancer model.

A [²²⁵Ac]-DOTA-anti-FGFR3 conjugate was synthesized using a pure R enantiomer of Compound C (see Example 4), MFGR1877S (vofatamab), and actinium-225.

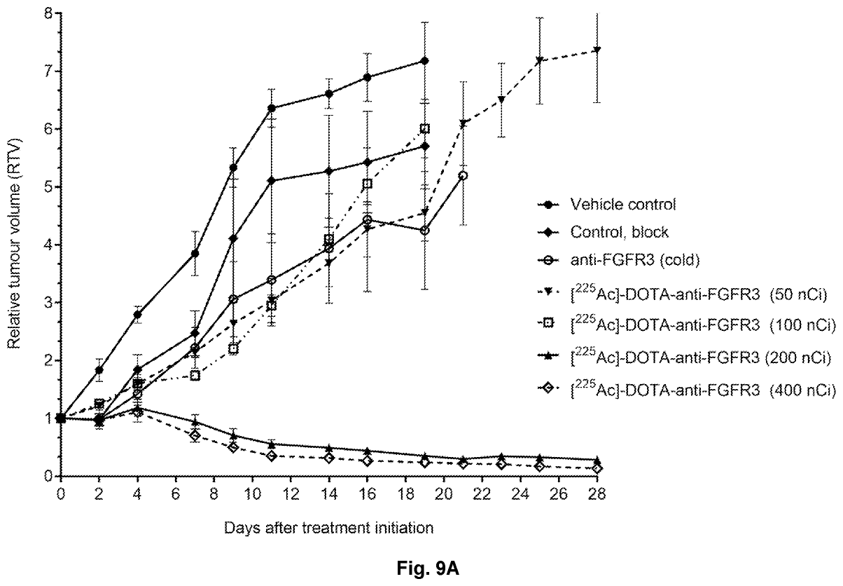

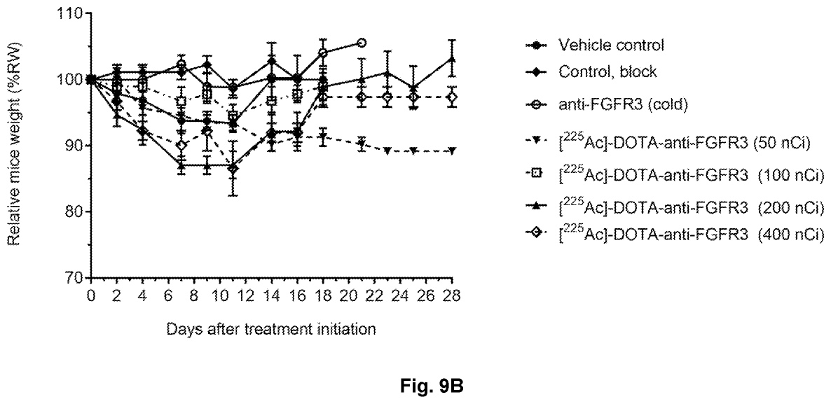

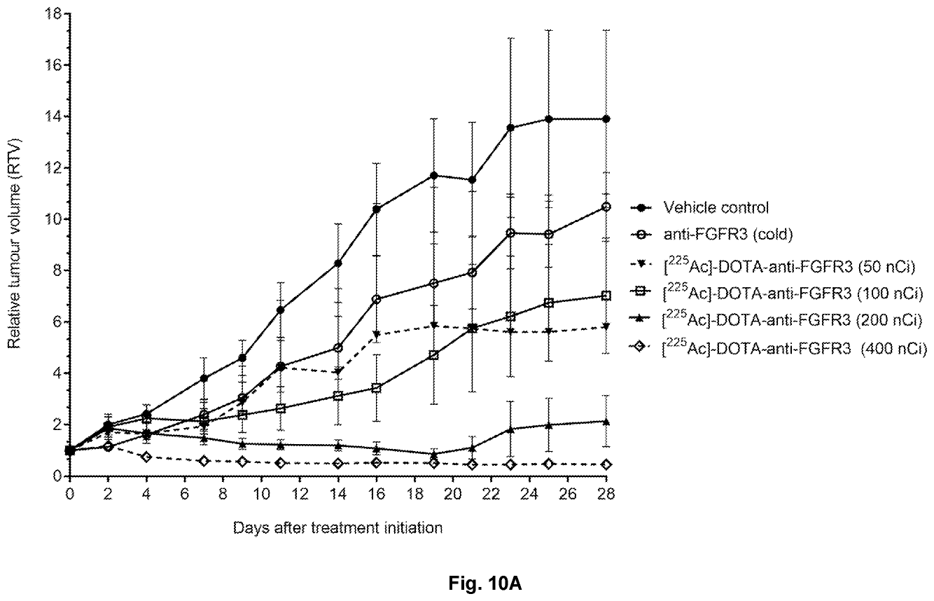

A Balb/c nude/RT112 cell line xenograft mouse model was used to assess the in vivo activity of [²²⁵Ac]-DOTA-anti-FGFR3 conjugate after pre-dosing with cold anti-FGFR3. Tumors were grown subcutaneously to about 150 mm³ in volume. Groups of tumor-bearing mice were injected intravenously with [²²⁵Ac]-DOTA-anti-FGFR3 (50 nCi, 100 nCi, 200 nCi, or 400 nCi doses), cold anti-FGFR3, or vehicle controls (n=5 per group). Except for mice in a control group, 3 hours before administration of [²²⁵Ac]-DOTA-anti-FGFR3, mice were injected intraperitoneally with 100 μg cold anti-FGFR3. Relative tumor volume (FIG. 9A) and relative body weights (FIG. 9B) were evaluated up to 28 days after administration.

As shown in FIG. 9A, treatment with 200 nCi or 400 nCi [²²⁵Ac]-DOTA-anti-FGFR3 significantly inhibited tumor growth. One mouse in the 400 nCi group lost 30% of its body weight and was sacrificed on Day 11. However, as shown in FIG. 9B, on average, mice in treatment groups did not demonstrate significant weight loss relative to mice in control groups, suggesting that the treatment was tolerated and that toxicity was limited.

### Example 21. Effects of [²²⁵Ac]-DOTA-Anti-FGFR3 Conjugates on Tumor Growth and Survival in a Bladder Cancer Xenograft Model

The present Example demonstrates therapeutic efficacy of an [²²⁵Ac]-DOTA-anti-FGFR3 conjugate in a bladder cancer model.

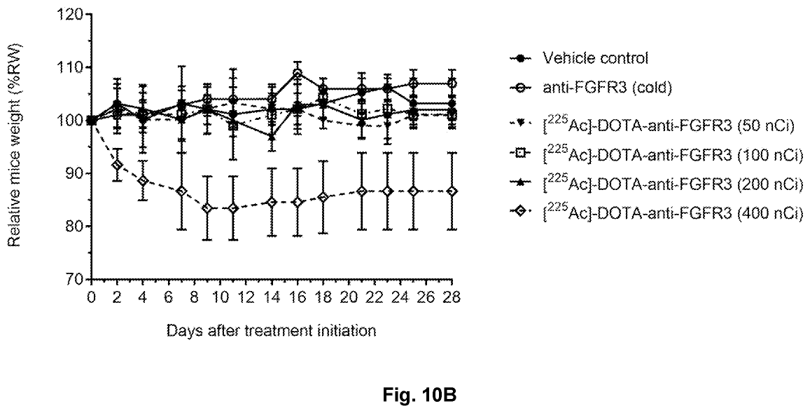

A Balb/c nude/RT112 cell line xenograft mouse model was used to assess the in vivo activity of [²²⁵Ac]-DOTA-anti-FGFR3 conjugate with co-dosing of cold anti-FGFR3. Tumors were allowed to grow subcutaneously to about 150 mm³ in volume. Groups of tumor bearing mice were injected intravenously with [²²⁵Ac]-DOTA-anti-FGFR3 (50 nCi, 100 nCi, 200 nCi, or 400 nCi) co-dosed with 100 μg anti-FGFR3. Control groups received cold anti-FGFR3 only or a vehicle control. n=5 per group. Relative tumor volume (FIG. 10A) and relative body weights (FIG. 10B) were evaluated up to 28 days after administration.

As shown in FIG. 10A, treatment with 200 nCi or 400 nCi [²²⁵Ac]-DOTA-anti-FGFR3 significantly inhibited tumor growth, and treatment with lower doses (50-100 nCi) of [²²⁵Ac]-DOTA-anti-FGFR3 resulted in some inhibition of tumor growth.

In the 400 nCi treatment group, two mice lost significant weight and were sacrificed, and the other three mice were not affected. However, mice in the other treatment groups did not demonstrate significant weight loss relative to mice in control groups. (See FIG. 10B.)

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features herein before set forth.

What is claimed is:

1. A radioimmunoconjugate comprising the following structure:
A-L-B Formula I-a
wherein A is a chelating moiety or metal complex thereof,
wherein B is an FGFR3 targeting moiety, and
wherein L is a linker.

2. The radioimmunoconjugate of claim 1, wherein A is a metal complex of a chelating moiety.

3. The radioimmunoconjugate of claim 2, wherein the metal complex comprises a radionuclide.

4. The radioimmunoconjugate of claim 3, wherein the radionuclide is an alpha emitter.

5. The radioimmunoconjugate of claim 4, wherein the radionuclide is an alpha emitter selected from the group consisting of Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), and Terbium-149 (¹⁴⁹Tb), or a progeny thereof.

6. The radioimmunoconjugate of claim 5, wherein the radionuclide is ²²⁵Ac or a progeny thereof.

7. The radioimmunoconjugate of claim 1, wherein L is has the structure -L¹-(L²)ₙ-, as shown within Formula I-b:
A-L¹-(L²)ₙ-B Formula I-b
wherein
A is a chelating moiety or metal complex thereof;
B is an FGFR3 targeting moiety;
L¹ is optionally substituted C₁-C₆ alkyl, optionally substituted C₁-C₆ heteroalkyl, or optionally substituted aryl or heteroaryl;
n is between 1 and 5 (inclusive); and
each L², independently, has the structure:
(—X¹-L³-Z¹—) Formula III
wherein
X¹ is C═O(NR¹), C═S(NR¹), OC═O(NR¹), NR¹C═O(O), NR¹C═O(NR¹), —CH₂PhC═O(NR¹), —CH₂Ph(NH)C═S(NR¹), O, or NR¹; and each R¹ independently is H, optionally substituted C₁-C₆ alkyl, optionally substituted C₁-C₆ heteroalkyl, or optionally substituted aryl or heteroaryl, in which C₁-C₆ alkyl can be substituted by oxo (═O), heteroaryl, or a combination thereof;
L³ is optionally substituted C₁-C₅₀ alkyl or optionally substituted C₁-C₅₀ heteroalkyl; and
Z¹ is CH₂, C═O, C═S, OC═O, NR¹C═O, or NR¹, wherein R¹ is a hydrogen or optionally substituted C₁-C₆ alkyl or pyrrolidine-2,5-dione.

8. The radioimmunoconjugate of claim 7, wherein L³ is C₅-C₂₀ polyethylene glycol.

9. The radioimmunoconjugate of claim 7, or a pharmaceutically acceptable salt thereof wherein the radioimmunoconjugate comprises the following structure: 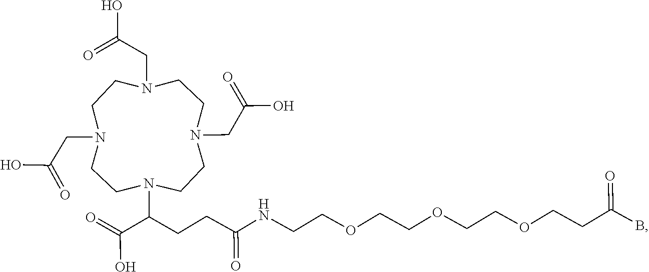
wherein B is an FGFR3 targeting moiety.

10. The radioimmunoconjugate of any one of claims 1-9, wherein the FGFR3 targeting moiety is at least 100 kDa in size.

11. The radioimmunoconjugate of claim 10, wherein the FGFR3 targeting moiety is at least 150 kDa in size.

12. The radioimmunoconjugate of claim 11, wherein the FGFR3 targeting moiety is at least 200 kDa in size.

13. The radioimmunoconjugate of claim 12, wherein the FGFR3 targeting moiety is at least 250 kDa in size.

14. The radioimmunoconjugate of claim 13, wherein the FGFR3 targeting moiety is at least 300 kDa in size.

15. The radioimmunoconjugate of any one of claims 1-14, wherein the FGFR3 targeting moiety is capable of binding to human FGFR3.

16. The radioimmunoconjugate of any one of claims 1-15, wherein the FGFR3 targeting moiety is capable of binding to wild type FGFR3.

17. The radioimmunoconjugate of any one of claims 1-15, wherein the FGFR3 targeting moiety is capable of binding to a mutant FGFR3.

18. The radioimmunoconjugate of any one of claim 17, wherein the FGFR3 targeting moiety is capable of binding to both wild type and a mutant FGFR3.

19. The radioimmunoconjugate of claim 17 or 18, wherein the mutant FGFR3 comprises a point mutation.

20. The radioimmunoconjugate of claim 19, wherein the point mutation is associated with cancer.

21. The radioimmunoconjugate of claim 20, wherein the point mutant is selected from the group consisting of FGFR3^{Y375C}, FGFR3^{R248C}, FGFR3^{S249C}, FGFR3^{G372C}, FGFR3^{K652E}, FGFR3^{K652Q}, FGFR3^{K652M}, and combinations thereof.

22. The radioimmunoconjugate of any one of claims 16-21, wherein the mutant FGFR3 comprises an FGFR3 fusion.

23. The radioimmunoconjugate of claim 22, wherein the FGFR3 fusion is selected from the group consisting of FGFR3-TACC3, FGFR3-CAMK2A, FGFR3-JAKMOP1, FGFR3-TNIP2, FGFR3-WHSC1, FGFR3-BAIAP2L1, and combinations thereof.

24. The radioimmunoconjugate of any one of claims 1-23, wherein the FGFR3 targeting moiety comprises an antibody or antigen-binding fragment thereof.

25. The radioimmunoconjugate of claim 24, wherein the antibody or antigen-binding fragment thereof is humanized.

26. The radioimmunoconjugate of claim 24 or 25, wherein the antibody or antigen-binding fragment thereof comprises at least one complementarity determining region (CDR) selected from the group consisting of:
CDR-H1 comprising the amino acid sequence of SEQ ID NO: 1, or an amino acid sequence differing in 1 or 2 amino acids therefrom;
CDR-H2 comprising the amino acid sequence of SEQ ID NO: 2, or an amino acid sequence differing in 1 or 2 amino acids therefrom;
CDR-H3 comprising the amino acid sequence of SEQ ID NO: 3 or 4, or an amino acid sequence differing in 1 or 2 amino acids from SEQ ID NO: 3 or 4;
CDR-L1 comprising the amino acid sequence of SEQ ID NO: 5, or an amino acid sequence differing in 1 or 2 amino acids therefrom;
CDR-L2 comprising the amino acid sequence of SEQ ID NO: 6, or an amino acid sequence differing in 1 or 2 amino acids therefrom; or
CDR-L3 comprising the amino acid sequence of SEQ ID NO: 7, or an amino acid sequence differing in 1 or 2 amino acids therefrom.

27. The radioimmunoconjugate of claim 26, wherein the antibody or antigen-binding fragment thereof comprises at least two CDRs selected from the group consisting of:
CDR-H1 comprising the amino acid sequence of SEQ ID NO: 1, or an amino acid sequence differing in 1 or 2 amino acids therefrom;
CDR-H2 comprising the amino acid sequence of SEQ ID NO: 2, or an amino acid sequence differing in 1 or 2 amino acids therefrom;
CDR-H3 comprising the amino acid sequence of SEQ ID NO: 3 or 4, or an amino acid sequence differing in 1 or 2 amino acids from SEQ ID NO: 3 or 4;
CDR-L1 comprising the amino acid sequence of SEQ ID NO: 5, or an amino acid sequence differing in 1 or 2 amino acids therefrom;
CDR-L2 comprising the amino acid sequence of SEQ ID NO: 6, or an amino acid sequence differing in 1 or 2 amino acids therefrom; or
CDR-L3 comprising the amino acid sequence of SEQ ID NO: 7, or an amino acid sequence differing in 1 or 2 amino acids therefrom.

28. The radioimmunoconjugate of claim 27, wherein the antibody or antigen-binding fragment thereof comprises at least three CDRs selected from the group consisting of:
CDR-H1 comprising the amino acid sequence of SEQ ID NO: 1, or an amino acid sequence differing in 1 or 2 amino acids therefrom;
CDR-H2 comprising the amino acid sequence of SEQ ID NO: 2, or an amino acid sequence differing in 1 or 2 amino acids therefrom;
CDR-H3 comprising the amino acid sequence of SEQ ID NO: 3 or 4, or an amino acid sequence differing in 1 or 2 amino acids from SEQ ID NO: 3 or 4;
CDR-L1 comprising the amino acid sequence of SEQ ID NO: 5, or an amino acid sequence differing in 1 or 2 amino acids therefrom;
CDR-L2 comprising the amino acid sequence of SEQ ID NO: 6, or an amino acid sequence differing in 1 or 2 amino acids therefrom; or
CDR-L3 comprising the amino acid sequence of SEQ ID NO: 7, or an amino acid sequence differing in 1 or 2 amino acids therefrom.

29. The radioimmunoconjugate of claim 28, wherein the antibody or antigen-binding fragment thereof comprises at least four CDRs selected from the group consisting of:
CDR-H1 comprising the amino acid sequence of SEQ ID NO: 1, or an amino acid sequence differing in 1 or 2 amino acids therefrom;
CDR-H2 comprising the amino acid sequence of SEQ ID NO: 2, or an amino acid sequence differing in 1 or 2 amino acids therefrom;
CDR-H3 comprising the amino acid sequence of SEQ ID NO: 3 or 4, or an amino acid sequence differing in 1 or 2 amino acids from SEQ ID NO: 3 or 4;
CDR-L1 comprising the amino acid sequence of SEQ ID NO: 5, or an amino acid sequence differing in 1 or 2 amino acids therefrom;
CDR-L2 comprising the amino acid sequence of SEQ ID NO: 6, or an amino acid sequence differing in 1 or 2 amino acids therefrom; or
CDR-L3 comprising the amino acid sequence of SEQ ID NO: 7, or an amino acid sequence differing in 1 or 2 amino acids therefrom.

30. The radioimmunoconjugate of claim 29, wherein the antibody or antigen-binding fragment thereof comprises at least five CDRs selected from the group consisting of:
CDR-H1 comprising the amino acid sequence of SEQ ID NO: 1, or an amino acid sequence differing in 1 or 2 amino acids therefrom;
CDR-H2 comprising the amino acid sequence of SEQ ID NO: 2, or an amino acid sequence differing in 1 or 2 amino acids therefrom;
CDR-H3 comprising the amino acid sequence of SEQ ID NO: 3 or 4, or an amino acid sequence differing in 1 or 2 amino acids from SEQ ID NO: 3 or 4;
CDR-L1 comprising the amino acid sequence of SEQ ID NO: 5, or an amino acid sequence differing in 1 or 2 amino acids therefrom;
CDR-L2 comprising the amino acid sequence of SEQ ID NO: 6, or an amino acid sequence differing in 1 or 2 amino acids therefrom; or
CDR-L3 comprising the amino acid sequence of SEQ ID NO: 7, or an amino acid sequence differing in 1 or 2 amino acids therefrom.

31. The radioimmunoconjugate of claim 30, wherein the antibody or antigen-binding fragment thereof comprises:
CDR-H1 comprising the amino acid sequence of SEQ ID NO: 1, or an amino acid sequence differing in 1 or 2 amino acids therefrom;
CDR-H2 comprising the amino acid sequence of SEQ ID NO: 2, or an amino acid sequence differing in 1 or 2 amino acids therefrom;
CDR-H3 comprising the amino acid sequence of SEQ ID NO: 3 or 4, or an amino acid sequence differing in 1 or 2 amino acids from SEQ ID NO: 3 or 4;
CDR-L1 comprising the amino acid sequence of SEQ ID NO: 5, or an amino acid sequence differing in 1 or 2 amino acids therefrom;
CDR-L2 comprising the amino acid sequence of SEQ ID NO: 6, or an amino acid sequence differing in 1 or 2 amino acids therefrom; and
CDR-L3 comprising the amino acid sequence of SEQ ID NO: 7, or an amino acid sequence differing in 1 or 2 amino acids therefrom.

32. The radioimmunoconjugate of claim 24 or 25, wherein the antibody or antigen-binding fragment thereof comprises
(i) a heavy chain variable domain comprising at least one CDR selected from the group consisting of:
CDR-H1 comprising the amino acid sequence of SEQ ID NO: 1, or an amino acid sequence differing in 1 or 2 amino acids therefrom;
CDR-H2 comprising the amino acid sequence of SEQ ID NO: 2, or an amino acid sequence differing in 1 or 2 amino acids therefrom; and
CDR-H3 comprising the amino acid sequence of SEQ ID NO: 3 or 4, or an amino acid sequence differing in 1 or 2 amino acids from SEQ ID NO: 3 or 4; and
(ii) a light chain variable domain comprising at least one CDR selected from the group consisting of:
CDR-L1 comprising the amino acid sequence of SEQ ID NO: 5, or an amino acid sequence differing in 1 or 2 amino acids therefrom;
CDR-L2 comprising the amino acid sequence of SEQ ID NO: 6, or an amino acid sequence differing in 1 or 2 amino acids therefrom; and
CDR-L3 comprising the amino acid sequence of SEQ ID NO: 7, or an amino acid sequence differing in 1 or 2 amino acids therefrom.

33. The radioimmunoconjugate of claim 32, wherein the antibody or antigen-binding fragment thereof comprises
(i) a heavy chain variable domain comprising at least one CDR selected from the group consisting of:
CDR-H1 comprising the amino acid sequence of SEQ ID NO: 1;
CDR-H2 comprising the amino acid sequence of SEQ ID NO: 2; and
CDR-H3 comprising the amino acid sequence of SEQ ID NO: 3 or 4; and
(ii) a light chain variable domain comprising at least one CDR selected from the group consisting of:
CDR-L1 comprising the amino acid sequence of SEQ ID NO: 5;
CDR-L2 comprising the amino acid sequence of SEQ ID NO: 6; and
CDR-L3 comprising the amino acid sequence of SEQ ID NO: 7.

34. The radioimmunoconjugate of claim 32 or 33, wherein the antibody or antigen-binding fragment thereof comprises
(i) a heavy chain variable domain comprising at least two CDRs selected from the group consisting of:
CDR-H1 comprising the amino acid sequence of SEQ ID NO: 1, or an amino acid sequence differing in 1 or 2 amino acids therefrom;
CDR-H2 comprising the amino acid sequence of SEQ ID NO: 2, or an amino acid sequence differing in 1 or 2 amino acids therefrom; and
CDR-H3 comprising the amino acid sequence of SEQ ID NO: 3 or 4, or an amino acid sequence differing in 1 or 2 amino acids from SEQ ID NO: 3 or 4; and
(ii) a light chain variable domain comprising at least two CDRs selected from the group consisting of:
CDR-L1 comprising the amino acid sequence of SEQ ID NO: 5, or an amino acid sequence differing in 1 or 2 amino acids therefrom;
CDR-L2 comprising the amino acid sequence of SEQ ID NO: 6, or an amino acid sequence differing in 1 or 2 amino acids therefrom; and
CDR-L3 comprising the amino acid sequence of SEQ ID NO: 7, or an amino acid sequence differing in 1 or 2 amino acids therefrom.

35. The radioimmunoconjugate of claim 34, wherein the antibody or antigen-binding fragment thereof comprises
(i) a heavy chain variable domain comprising at least two CDRs selected from the group consisting of:
CDR-H1 comprising the amino acid sequence of SEQ ID NO: 1;
CDR-H2 comprising the amino acid sequence of SEQ ID NO: 2; and
CDR-H3 comprising the amino acid sequence of SEQ ID NO: 3 or 4; and
(ii) a light chain variable domain comprising at least two CDRs selected from the group consisting of:
CDR-L1 comprising the amino acid sequence of SEQ ID NO: 5;
CDR-L2 comprising the amino acid sequence of SEQ ID NO: 6; and
CDR-L3 comprising the amino acid sequence of SEQ ID NO: 7.

36. The radioimmunoconjugate of claim 35, wherein the antibody or antigen-binding fragment thereof comprises
(i) a heavy chain variable domain comprising:
CDR-H1 comprising the amino acid sequence of SEQ ID NO: 1, or an amino acid sequence differing in 1 or 2 amino acids therefrom;
CDR-H2 comprising the amino acid sequence of SEQ ID NO: 2, or an amino acid sequence differing in 1 or 2 amino acids therefrom; and
CDR-H3 comprising the amino acid sequence of SEQ ID NO: 3 or 4, or an amino acid sequence differing in 1 or 2 amino acids from SEQ ID NO: 3 or 4; and
(ii) a light chain variable domain comprising:
CDR-L1 comprising the amino acid sequence of SEQ ID NO: 5, or an amino acid sequence differing in 1 or 2 amino acids therefrom;
CDR-L2 comprising the amino acid sequence of SEQ ID NO: 6, or an amino acid sequence differing in 1 or 2 amino acids therefrom; and
CDR-L3 comprising the amino acid sequence of SEQ ID NO: 7, or an amino acid sequence differing in 1 or 2 amino acids therefrom.

37. The radioimmunoconjugate of claim 36, wherein the antibody or antigen-binding fragment thereof comprises
(i) a heavy chain variable domain comprising:
CDR-H1 comprising the amino acid sequence of SEQ ID NO: 1;
CDR-H2 comprising the amino acid sequence of SEQ ID NO: 2; and
CDR-H3 comprising the amino acid sequence of SEQ ID NO: 3 or 4; and
(ii) a light chain variable domain comprising:
CDR-L1 comprising the amino acid sequence of SEQ ID NO: 5;
CDR-L2 comprising the amino acid sequence of SEQ ID NO: 6; and
CDR-L3 comprising the amino acid sequence of SEQ ID NO: 7.

38. The radioimmunoconjugate of any one of claims 27-37, wherein the antibody or antigen-binding fragment thereof comprises
(i) a heavy chain variable domain having an amino acid sequence with at least 85% identity with the amino acid sequence of SEQ ID NO: 8; and
(ii) a light chain variable domain having an amino acid sequence with at least 85% identity with the amino acid sequence of SEQ ID NO: 9.

39. The radioimmunoconjugate of claim 38, wherein the antibody or antigen-binding fragment thereof comprises
(i) a heavy chain variable domain having an amino acid sequence with at least 90% identity with the amino acid sequence of SEQ ID NO: 8; and
(ii) a light chain variable domain having an amino acid sequence with at least 90% identity with the amino acid sequence of SEQ ID NO: 9.

40. The radioimmunoconjugate of claim 39, wherein the antibody or antigen-binding fragment thereof comprises
(i) a heavy chain variable domain having an amino acid sequence with at least 95% identity with the amino acid sequence of SEQ ID NO: 8; and
(ii) a light chain variable domain having an amino acid sequence with at least 95% identity with the amino acid sequence of SEQ ID NO: 9.

41. The radioimmunoconjugate of claim 40, wherein the antibody or antigen-binding fragment thereof comprises
(i) a heavy chain variable domain comprising the amino acid sequence of SEQ ID NO: 8; and
(ii) a light chain variable domain comprising the amino acid sequence of SEQ ID NO: 9.

42. The radioimmunoconjugate of claim 41, wherein the antibody is MFGR1877S (vofatamab).

43. The radioimmunoconjugate of any one of claims 1-42, wherein, after administration of the radioimmunoconjugate or a composition thereof to a mammal, the proportion of radiation excreted by the intestinal routes, renal route, or both routes is at least 2-fold greater than the proportion of radiation excreted by the same route(s) by a comparable mammal that has been administered a reference radioimmunoconjugate.

44. The radioimmunoconjugate of claim 43, wherein, after administration of the radioimmunoconjugate or a composition thereof to a mammal, the proportion of radiation excreted by the intestinal routes, renal route, or both routes is at least 3-fold greater than the proportion of radiation excreted by the same route(s) by a comparable mammal that has been administered a reference radioimmunoconjugate.

45. The radioimmunoconjugate of claim 1, wherein A-L- is a metal complex of a compound selected from the group consisting of:

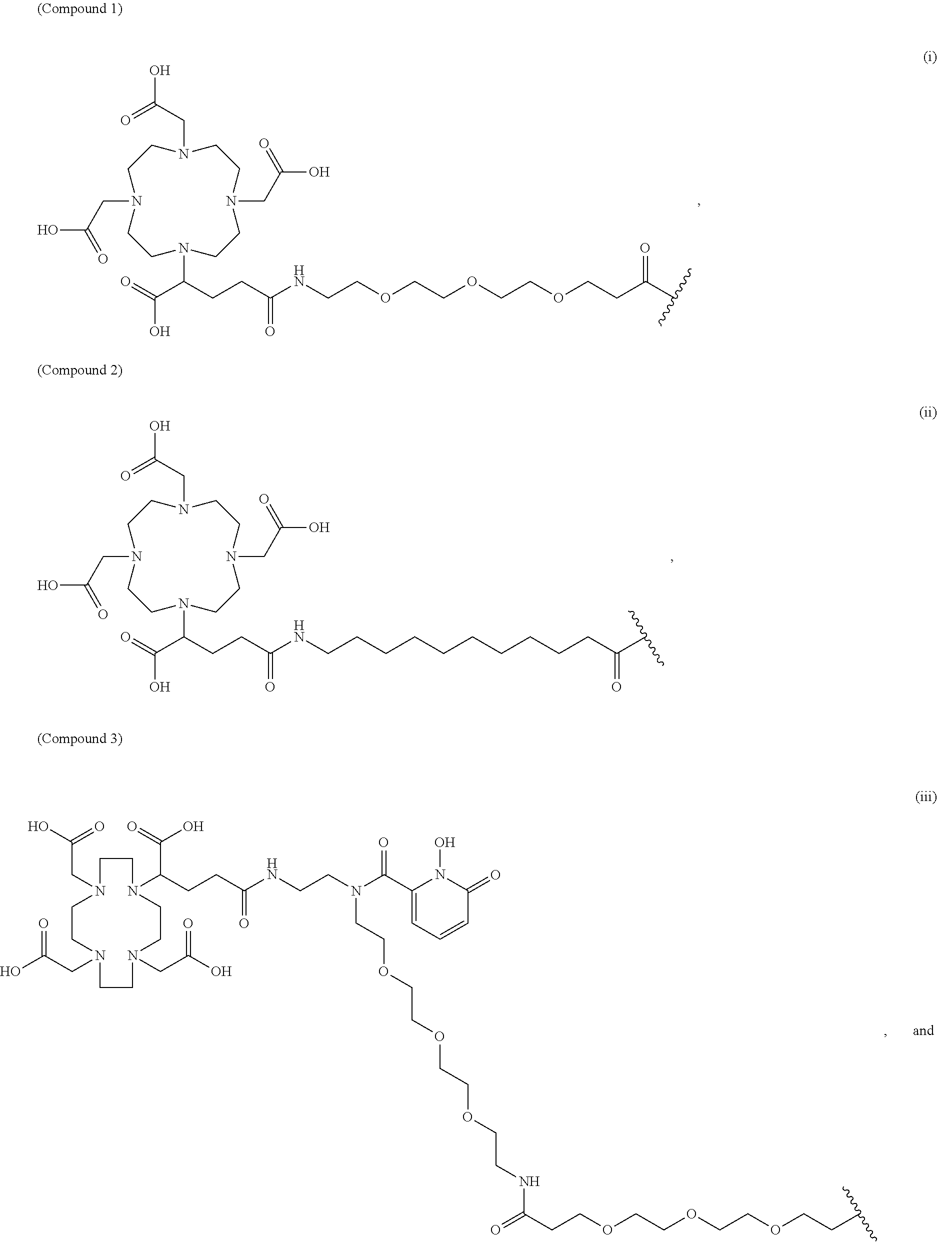 [SMILES: *C(=O)CCOCCOCCOCCNC(=O)CCC(C(=O)O)N1CCN(CC(=O)O)CCN(CC(=O)O)CCN(CC(=O)O)CC1 ; *C(=O)CCCCCCCCCCNC(=O)CCC(C(=O)O)N1CCN(CC(=O)O)CCN(CC(=O)O)CCN(CC(=O)O)CC1 ; *CCOCCOCCOCCC(=O)NCCOCCOCCOCCN(CCNC(=O)CCC(C(=O)O)N1CCN(CC(=O)O)CCN(CC(=O)O)CCN(CC(=O)O)CC1)C(=O)c1cccc(=O)n1O]

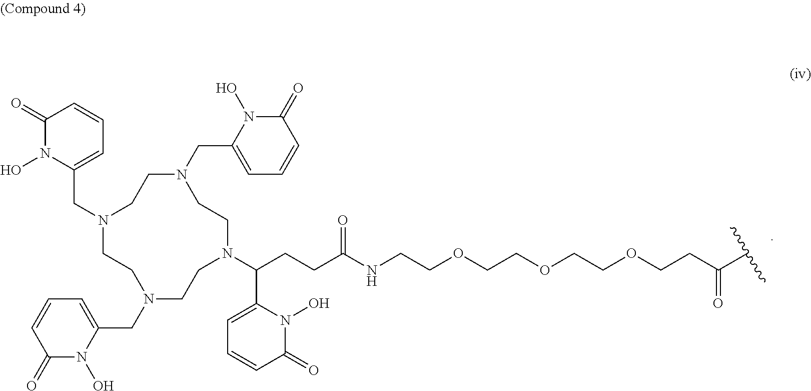 [SMILES: *C(=O)CCOCCOCCOCCNC(=O)CCC(c1cccc(=O)n1O)N1CCN(Cc2cccc(=O)n2O)CCN(Cc2cccc(=O)n2O)CCN(Cc2cccc(=O)n2O)CC1]

46. The radioimmunoconjugate of claim 45, wherein A-L- is a metal complex of:

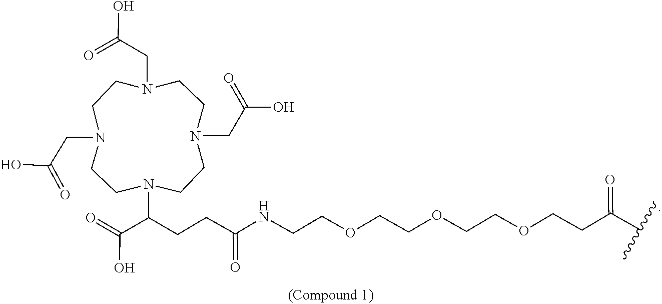 [SMILES: *C(=O)CCOCCOCCOCCNC(=O)CCC(C(=O)O)N1CCN(CC(=O)O)CCN(CC(=O)O)CCN(CC(=O)O)CC1]

47. The radioimmunoconjugate of claim 46, wherein the metal complex comprises a radionuclide.

48. The radioimmunoconjugate of claim 47, wherein the radionuclide is an alpha emitter.

49. The radioimmunoconjugate of claim 48, wherein the radionuclide is an alpha emitter selected from the group consisting of Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), and Terbium-149 (¹⁴⁹Tb), or a progeny thereof.

50. The radioimmunoconjugate of claim 49, wherein the radionuclide is ²²⁵Ac or a progeny thereof.

51. The radioimmunoconjugate of claim 50, wherein the FGFR3 targeting moiety comprises an antibody or antigen-binding fragment thereof.

52. The radioimmunoconjugate of claim 51, wherein the antibody or antigen-binding fragment thereof is humanized.

53. The radioimmunoconjugate of claim 52, wherein the antibody or antigen-binding fragment thereof comprises
(i) a heavy chain variable domain comprising:
CDR-H1 comprising the amino acid sequence of SEQ ID NO: 1;
CDR-H2 comprising the amino acid sequence of SEQ ID NO: 2; and
CDR-H3 comprising the amino acid sequence of SEQ ID NO: 3 or 4; and
(ii) a light chain variable domain comprising:
CDR-L1 comprising the amino acid sequence of SEQ ID NO: 5;
CDR-L2 comprising the amino acid sequence of SEQ ID NO: 6; and
CDR-L3 comprising the amino acid sequence of SEQ ID NO: 7.

54. The radioimmunoconjugate of claim 52, wherein the antibody or antigen-binding fragment thereof comprises
(i) a heavy chain variable domain having an amino acid sequence with at least 95% identity with the amino acid sequence of SEQ ID NO: 8; and
(ii) a light chain variable domain having an amino acid sequence with at least 95% identity with the amino acid sequence of SEQ ID NO: 9.

55. The radioimmunoconjugate of claim 54, wherein the antibody or antigen-binding fragment thereof comprises
(i) a heavy chain variable domain comprising the amino acid sequence of SEQ ID NO: 8; and
(ii) a light chain variable domain comprising the amino acid sequence of SEQ ID NO: 9.

56. The radioimmunoconjugate of claim 55, wherein the antibody or antigen-binding fragment thereof is MFGR1877S (vofatamab) or an antigen-binding fragment thereof.

57. The radioimmunoconjugate of claim 56, wherein the antibody or antigen-binding fragment thereof is MFGR1877S (vofatamab).

58. The radioimmunoconjugate of claim 46, said radioimmunoconjugate comprising the following structure: 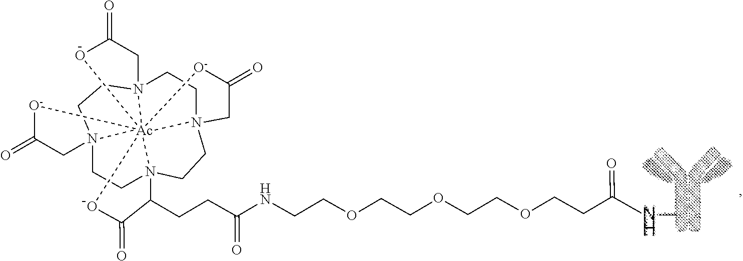
wherein [symbol] is MFGR1877S (vofatamab), wherein the amine group NH— attached to the antibody shown above is from a lysine unit that is part of the antibody.

59. A pharmaceutical composition comprising the radioimmunoconjugate of any one of claims 1-58 and a pharmaceutically acceptable carrier.

60. A method of treating cancer, the method comprising administering to a subject in need thereof a pharmaceutical composition comprising an effective amount of the radioimmunoconjugate of any one of claims 1-58.

61. The method of claim 60, wherein the subject is a mammal.

62. The method of claim 61, wherein the mammal is a human.

63. The method of any one of claims 60-62, wherein the cancer is a solid tumor cancer.

64. The method of claim 63, wherein the solid tumor cancer is adrenocortical carcinoma, bladder cancer, breast cancer, cervical cancer, colorectal cancer, endometrial adenocarcinoma, Ewing's sarcoma, gallbladder carcinoma, glioma, head and neck cancer, liver cancer, lung cancer, neuroblastoma, neuroendocrine cancer, pancreatic cancer, prostate cancer, renal cell carcinoma, salivary adenoid cystic cancer, or spermatocytic seminoma.

65. The method of claim 64, wherein the solid tumor cancer is bladder cancer.

66. The method of claim 64, wherein the solid tumor cancer is glioma.

67. The method of claim 64, wherein the solid tumor cancer is neuroblastoma.

68. The method of claim 64, wherein the solid tumor cancer is pancreatic cancer.

69. The method of claim 64, wherein the solid tumor cancer is breast cancer.

70. The method of claim 64, wherein the solid tumor cancer is head and neck cancer.

71. The method of claim 64, wherein the solid tumor cancer is liver cancer.

72. The method of claim 64, wherein the solid tumor cancer is lung cancer.

73. The method of any one of claims 60-62, wherein the cancer is a non-solid tumor cancer.

74. The method of claim 73, wherein the cancer is a liquid cancer or hematologic cancer.

75. The method of claim 74, wherein the cancer is a myeloma.

76. The method of claim 75, wherein the myeloma is multiple myeloma.

77. The method of claim 74, wherein the cancer is a leukemia.

78. The method of claim 74, wherein the cancer is lymphoma.

79. The method of any one of claims 60-78, wherein the pharmaceutical composition is administered systemically.

80. The method of claim 79, wherein the pharmaceutical composition is administered parenterally.

81. The method of claim 80, wherein the pharmaceutical composition is administered intravenously.

82. The method of claim 80, wherein the pharmaceutical composition is administered intraarterially.

83. The method of claim 80, wherein the pharmaceutical composition is administered intraperitoneally.

84. The method of claim 80, wherein the pharmaceutical composition is administered subcutaneously.

85. The method of claim 80, wherein the pharmaceutical composition is administered intradermally.

86. The method of claim 79, wherein the pharmaceutical composition is administered enterically.

87. The method of claim 86, wherein the pharmaceutical composition is administered trans-gastrointestinally.

88. The method of claim 86, wherein the pharmaceutical composition is administered orally.

89. The method of any one of claims 60-78, wherein the pharmaceutical composition is administered locally.

90. The method of claim 89, wherein the pharmaceutical composition is administered by peritumoral injection.

91. The method of claim 89, wherein the pharmaceutical composition is administered by intratumoral injection.


SEQUENCE LISTING (9 sequences)

SEQ ID NO: 1 - protein, 10 aa, Artificial Sequence
  CDR-H1 of FGFR3 antibody
GFTFTSTGIS

SEQ ID NO: 2 - protein, 16 aa, Artificial Sequence
  CDR-H2 of FGFR3 antibody
GRIYPTSGSTNYADSV

SEQ ID NO: 3 - protein, 18 aa, Artificial Sequence
  CDR-H3 of FGFR3 antibody
TYGIYDLYVDYTEYVMDY

SEQ ID NO: 4 - protein, 20 aa, Artificial Sequence
  CDR-H3 of FGFR3 antibody
ARTYGIYDLYVDYTEYVMDY

SEQ ID NO: 5 - protein, 11 aa, Artificial Sequence
  CDR-L1 of FGFR3 antibody
RASQDVDTSLA

SEQ ID NO: 6 - protein, 7 aa, Artificial Sequence
  CDR-L2 of FGFR3 antibody
SASFLYS

SEQ ID NO: 7 - protein, 9 aa, Artificial Sequence
  CDR-L3 of FGFR3 antibody
QQSTGHPQT

SEQ ID NO: 8 - protein, 457 aa, Artificial Sequence
  Heavy chain variable sequence of FGFR3 antibody MFGR1877S
EVQLVESGGGLVQPGGSLRLSCAASGFTFTSTGISWVRQAPGKGLEWVGRIYPTSGSTNYADSVKGRFTISADTSKNTAYLQMNSLRAEDTAVYYCARTYGIYDLYVDYTEYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 214 aa, Artificial Sequence
  Light chain variable region of FGFR3 antibody MFGR1877S
DIQMTQSPSSLSASVGDRVTITCRASQDVDTSLAWYKQKPGKAPKLLIYSASFLYSGVPSRFSGSGSGTDFTLTISSLQPEDFATYYCQQSTGHPQTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC